(19) United States
(12) Patent Application Publication
(10) Pub. No.: US 2022/0106551 A1
(43) Pub. Date: Apr. 7, 2022

(54) CELL CULTURE SUBSTRATE

(71) Applicant: DAI NIPPON PRINTING CO., LTD., Tokyo (JP)

(72) Inventors: Yuichi TANAKA, Tokyo (JP); Koji FUJIMOTO, Tokyo (JP); Natsuki KAKEGAWA, Tokyo (JP)

(73) Assignee: DAI NIPPON PRINTING CO., LTD., Tokyo (JP)

(21) Appl. No.: 17/426,566

(22) PCT Filed: Jan. 29, 2020
(86) PCT No.: PCT/JP2020/003259

§ 371 Date: Jul. 28, 2021

(30) Foreign Application Priority Data
Jan. 30, 2019 (JP) 2019-014820

(51) Int. Cl. C12M 1/12; C12M 1/00; C12M 3/00; C12N 5/09; C12N 5/074

(52) U.S. Cl. CPC C12M 25/10; C12M 23/20; C12N 5/0696; C12N 5/0693; C12M 21/08

(57) ABSTRACT

A cell culture substrate having surface S including cell culture part, wherein the cell culture part includes non-cell-adhesive part, and cell-adhesive part extending continuously or intermittently along periphery P of the non-cell-adhesive part and surrounding the non-cell-adhesive part. The present disclosure also relates to a kit for cell culture comprising this cell culture substrate.

### TECHNICAL FIELD

The present disclosure relates to a substrate and a kit for culturing cells.

### BACKGROUND ART

As pluripotent stem cells such as embryonic stem cells (ES cells) and induced pluripotent stem cells (iPS cells) can be induced to differentiate into cells of interest, clinical applications of pluripotent stem cells in the field of regenerative medicine are expected. Methods for obtaining a structure called organoid similar to human tissues by culturing pluripotent stem cells to induce their differentiation are under study.

For example, Patent Literature 1 describes a method for producing an intestinal epithelial organoid from embryonic stem cells.

As disclosed in Non Patent Literature 2, the technique of inducing non-epithelial cells from an organoid by administering TNF-α into a medium has also been established.

Other examples of the method for inducing the differentiation of induced pluripotent stem cells into small intestinal epithelial cells include methods described in Patent Literature 2.

Meanwhile, Patent Literature 3 and Non Patent Literature 3 each describe a method for inducing differentiation into an intestinal tissue by culturing pluripotent stem cells on a substrate on which a pattern of a cell-adhesive part is formed.

Cancer cells are known to form cysts in vivo. The cysts have a spherical structure that contains liquid components pathologically formed in soft tissues where the periphery of the liquid components is covered with intrinsic single-layered epithelium. As a method for culturing a cyst-like cell construct from cancer cells ex vivo, a method involving gel-embedded three-dimensional culture is known (e.g., Non Patent Literature 11).

### CITATION LIST

### Patent Literature

Patent Literature 1: WO2011/140441
Patent Literature 2: JP Patent No. 6296399
Patent Literature 3: JP Patent No. 6151097
Patent Literature 4: JP Patent No. 5070565
Patent Literature 5: JP Patent Publication (Kokai) No. 2013-212088 A (2013)
Patent Literature 6: JP Patent No. 5140946

### Non Patent Literature

Non Patent Literature 1: Workman et al., Nature Medicine, Vol. 23, p. 49-59, 2017
Non Patent Literature 2: Hahn et al., Scientific Reports, Vol. 7, 2435, 2017
Non Patent Literature 3: Uchida et al., JCI Insight, Vol. 2, e86492, 2017
Non Patent Literature 4: Lee et al., Stem Cell Reports, Vol. 6, p. 257-272, 2016
Non Patent Literature 5: Okeyo et al., Tissue Eng Part C, Vol. 21, p. 1105-1115, 2015
Non Patent Literature 6: Golos et al., Placenta, Vol. 34S, p. S56-S61, 2013
Non Patent Literature 7: Chen et al., Biochemical and Biophysical Research Communications, Vol. 436, p. 677-684, 2013
Non Patent Literature 8: Kojima et al., Laboratory Investigation, Vol. 97, p. 1188-1200, 2017
Non Patent Literature 9: Niwa et al., Nature Genetics, Vol. 24, p. 372-376, 2000
Non Patent Literature 10: Okochi et al., Langmuir, Vol. 25, p. 6947-6953, 2009
Non Patent Literature 11: Spence et al., Nature, Vol. 470, p. 105-109, 2010

### SUMMARY OF INVENTION

### Technical Problem

The present disclosure provides means that enable production of a cell construct such as an intestinal tissue-like organoid or a cancer tissue- or cyst-like cell construct by cell culture.

### Solution to Problem

One or more embodiments of the present disclosure relate to

a cell culture substrate having a surface comprising a cell culture part, wherein

the cell culture part comprises a non-cell-adhesive part, and a cell-adhesive part extending continuously or intermittently along the periphery of the non-cell-adhesive part and surrounding the non-cell-adhesive part.

One or more embodiments of the present disclosure relate to

a cell culture substrate comprising a support substrate having a surface comprising

a first non-cell-adhesive part, and

one or more cell culture parts arranged in the first non-cell-adhesive part, wherein

each of the one or more cell culture parts comprises a central part serving as a second non-cell-adhesive part, and a cell-adhesive part extending continuously or intermittently along the periphery of the central part and surrounding the central part.

The cell culture substrate is preferably a cell culture substrate for culturing cells to induce a sac-shaped cell construct. The cells are preferably stem cells or cancer cells.

The cell culture substrate according to one or more embodiments is preferably a cell culture substrate for culturing stem cells to induce their differentiation into a cell construct containing small intestinal epithelial cells.

In a preferable embodiment of the cell culture substrate, a distance between two points of intersection of a straight line with an inner circumference of the cell-adhesive part is larger than 80 μm and 880 μm or smaller, more preferably 180 μm or larger and 600 μm or smaller, particularly preferably 180 μm or larger and 500 μm or smaller, the straight line passing through a midpoint between two points on the inner circumference of the cell-adhesive part opposed to and most distant from each other across the non-cell-adhesive part or the central part.

In a preferable embodiment of the cell culture substrate, a width of the cell-adhesive part along a straight line is larger than 30 μm and 400 μm or smaller, more preferably 40 μm or larger and 400 μm or smaller, further preferably 60 μm or larger and 300 μm or smaller, the straight line passing through a midpoint between two points on an inner circumference of the cell-adhesive part opposed to and most distant from each other across the non-cell-adhesive part or the central part.

In a preferable embodiment of the cell culture substrate, ratio X′/W′ of distance X′ to width W′ is preferably 0.5 or more, more preferably 1.0 or more, and further preferably 1.3 or more, and is preferably 20.0 or less, more preferably 15.0 or less, and further preferably 10.0 or less,

wherein the distance X is a distance between two points of intersection of a straight line with an inner circumference of the cell-adhesive part, the straight line passing through a midpoint between two points on the inner circumference of the cell-adhesive part opposed to and most distant from each other across the non-cell-adhesive part or the central part, and wherein the width W′ is a width of the cell-adhesive part along a straight line passing through the midpoint.

In a preferable embodiment of the cell culture substrate, a distance between two points of intersection of the periphery of the non-cell-adhesive part or the central part with a straight line passing through the barycenter of the non-cell-adhesive part or the central part is larger than 80 μm and 880 μm or smaller, more preferably 180 μm or larger and 600 μm or smaller, particularly preferably 180 μm or larger and 500 μm or smaller.

In a preferable embodiment of the cell culture substrate, a width of the cell-adhesive part along the straight line passing through the barycenter of the non-cell-adhesive part or the central part is larger than 30 μm and 400 μm or smaller, more preferably 40 μm or larger and 400 μm or smaller, further preferably 60 μm or larger and 300 μm or smaller.

In a preferable embodiment of the cell culture substrate, ratio X/W of distance X between two points of intersection of the periphery of the non-cell-adhesive part or the central part to a straight line passing through the barycenter of the non-cell-adhesive part or the central part, to width W of the cell-adhesive part along a straight line passing through the barycenter of the non-cell-adhesive part or the central part is preferably 0.5 or more, more preferably 1.0 or more, and further preferably 1.3 or more, and is preferably 20.0 or less, more preferably 15.0 or less, and further preferably 10.0 or less.

In a preferable embodiment of the cell culture substrate, the non-cell-adhesive part (which may be the first non-cell-adhesive part, the second non-cell-adhesive part or the central part) is a surface coated with a layer comprising a hydrophilic polymer. The hydrophilic polymer is preferably one or more hydrophilic polymers selected from polyalkylene glycol and a zwitterionic polymer having a phospholipid polar group. The polyalkylene glycol may be polyethylene glycol.

In a preferable embodiment of the cell culture substrate, the cell culture substrate comprises a glass substrate as a support substrate.

Alternative one or more embodiments of the present disclosure relate to

a kit for cell culture comprising the cell culture substrate.

In a preferable embodiment of the kit, the kit further comprises one or more members selected from a medium and a precoating treatment agent.

In a preferable embodiment of the kit, the kit is a kit for culturing cells to induce a sac-shaped cell construct. The cells are preferably stem cells or cancer cells.

Alternative one or more embodiments of the present disclosure relate to

a method for producing a sac-shaped cell construct, comprising:

seeding cells onto the cell culture substrate; and

culturing the cells seeded to induce a sac-shaped cell construct.

The cells are preferably stem cells or cancer cells.

Alternative one or more embodiments of the present disclosure relate to a sac-shaped cell construct produced by the production method.

Alternative one or more embodiments of the present disclosure relate to

a method for producing a cell construct comprising small intestinal epithelial cells, comprising:

seeding stem cells onto the cell culture substrate; and

culturing the stem cells seeded to differentiate a part of the stem cells into small intestinal epithelial cells.

Alternative one or more embodiments of the present disclosure relate to a cell construct comprising small intestinal epithelial cells produced by the production method.

The cell construct preferably comprises endodermal cells, ectodermal cells and mesodermal cells.

The present specification encompasses the contents disclosed in Japanese Patent Application No. 2019-014820 on which the priority of the present application is based.

### Advantageous Effects of Invention

A cell construct can be produced by use of the cell culture substrate or the kit of the present disclosure.

### BRIEF DESCRIPTION OF DRAWINGS

FIG. 1 is a schematic view of an embodiment in which a cell-adhesive part is an exposed surface of a support substrate, of a cell culture substrate having a plurality of annular cell-adhesive parts used in Example 1, Example 8 and Example 10. FIG. 1(A) is a plane view of the cell culture substrate, and FIG. 1(B) is a cross-sectional schematic view taken along the A-A line in FIG. 1(A).

FIG. 2 shows observation images of cultures on culture days 1, 6, 11 and 18 obtained by culture using each cell culture substrate having an annular cell-adhesive part having a different inside diameter in Example 1.

FIG. 3 shows observation images of cultures on culture week 3 obtained by culture using each cell culture substrate having an annular cell-adhesive part having a different inside diameter in Example 1.

FIG. 4 shows observation images of a tissue having a sac-shaped structure formed on and detached from a substrate having an annular cell-adhesive part having an inside diameter of 380 μm (the left photograph shows the whole dish, and the right photograph is an observation image of the tissue).

FIG. 5 shows microscopic observation images of cultures on 3 weeks after the start of culture on a substrate in each of Comparative Example 1, Comparative Example 2, and Comparative Example 3.

FIG. 6 shows observation images of cells around one annular cell-adhesive part on each of culture days 4, 9, 13 and 20 in Example 2.

FIG. 7 shows observation images on culture days 1 and 7 of cultures obtained by culture using cell culture substrates differing in inside diameter in Example 3, and observation images of tissues having a sac-shaped structure collected after 3-week culture using a cell culture substrate having an annular cell-adhesive part having an inside diameter of 280 μm.

FIG. 8 shows observation images on culture day 4 of immunostained cultures in Example 4.

FIG. 9 shows observation images on culture day 7 of immunostained cultures in Example 4.

FIG. 10 shows results of staining, with an anti-CDX2 antibody, an anti-villin antibody and DAPI, of tissues formed by culturing stem cells on a cell culture substrate having an annular cell-adhesive part having an inside diameter of 280 μm or 380 μm and a width of 60 μm in Example 5.

FIG. 11 shows results of staining, with an anti-smooth muscle actin antibody, an anti-PGP9.5 antibody and DAPI, of tissues formed by culture on a cell culture substrate having an annular cell-adhesive part having an inside diameter of 380 μm and a width of 60 μm in Example 5.

FIG. 12 shows observation images on day 18 of culture on a cell culture substrate having an annular cell-adhesive part of each size in Example 6.

FIG. 13 shows results of evaluating culture on a cell culture substrate having an annular cell-adhesive part of each size in Example 6.

FIG. 14 shows an observation image of a tissue having a sac-shaped structure obtained by culture on a cell culture substrate having an annular cell-adhesive part having an inside diameter of 580 μm and a width of 60 μm in Example 6.

FIG. 15A shows a square cell-adhesive part having an inside dimension of 280 μm to 300 μm in side and a width of 50 μm to 60 μm used in Example 7 and Example 10.

FIG. 15B shows an annular cell-adhesive part having an inside diameter of 280 μm and a width of 60 μm and lacking ⅛ in the circumferential direction used in Example 7 and Example 10.

FIG. 15C shows a rectangular cell-adhesive part having an inside dimension of 600 μm in long side and 300 μm in short side and a width of 50 μm used in Example 7.

FIG. 15D shows a square cell-adhesive part having an inside dimension of 600 μm in side (Example 7) and a width of 50 μm used in Example 7.

FIG. 16A shows observation images of a culture obtained by culturing stem cells using a cell culture substrate having a cell-adhesive part in the shape shown in FIG. 15A.

FIG. 16B shows observation images of a culture obtained by culturing stem cells using a cell culture substrate having a cell-adhesive part in the shape shown in FIG. 15B.

FIG. 16C shows observation images of a culture obtained by culturing stem cells using a cell culture substrate having a cell-adhesive part in the shape shown in FIG. 15C.

FIG. 16D shows observation images of a culture obtained by culturing stem cells using a cell culture substrate having a cell-adhesive part in the shape shown in FIG. 15D.

FIG. 17A shows cell culture substrate 100 having a surface having non-cell-adhesive region 101, and a plurality of linear cell-adhesive regions 102 of 30 to 50 μm in width arranged in parallel at intervals of 200 μm in the non-cell-adhesive region 101, used in Comparative Example 4.

FIG. 17B shows cell culture substrate 100′ having a surface having non-cell-adhesive region 101′, and a plurality of arc-like cell-adhesive regions 102′ of 50 μm in width lacking over a half in the circumferential direction of a ring having an inside diameter of 600 μm arranged in the non-cell-adhesive region 101′, used in Comparative Example 5.

FIG. 17C shows photographs on days 1 and 20 of cell culture on cell culture substrate 100 in Comparative Example 4.

FIG. 17D shows photographs on days 1 and 20 of cell culture on cell culture substrate 100′ in Comparative Example 5.

FIG. 18 shows observation images on culture days 1, 7 and 11 of cultures obtained by culturing stem cells on a cell culture substrate having an annular cell-adhesive part having an inside diameter of 280 μm or 380 μm and a width of 60 μm in Example 8.

FIG. 19 shows observation images of tissues having a sac-shaped structure obtained by culturing stem cells for 3 weeks on a cell culture substrate having an annular cell-adhesive part having an inside diameter of 280 μm or 380 μm and a width of 60 μm in Example 8.

FIG. 20 The photograph of “Example” in FIG. 20 is a typical photograph of a sac-shaped cell construct formed by culturing iPS cells (Nippon Genetics Co., Ltd.) established from human fibroblasts on a cell culture substrate having a plurality of annular cell-adhesive parts having a size of 600 μm in inside diameter and 100 μm in width in Example 9. The photograph of “Comparative Example” in FIG. 20 is a typical photograph of a sac-shaped cell construct formed by culturing the cells on a cell culture substrate of Comparative Example 1 having a plurality of round cell-adhesive parts of 1500 μm in diameter in Example 9.

FIG. 21 is a schematic view of an embodiment in which a cell-adhesive part is a surface of cell-adhesive layer, of a cell culture substrate having a plurality of annular cell-adhesive parts. FIG. 21(A) is a plane view of the cell culture substrate, and FIG. 21(B) is a cross-sectional schematic view taken along the A-A line in FIG. 21(A).

FIG. 22 shows a semicircular arc-like cell-adhesive part having an inside diameter of 280 μm and a width of 60 μm and lacking ½ in the circumferential direction of a ring used in Example 10.

FIG. 23 shows an observation image of a culture on culture day 18 obtained by culturing large intestinal epithelial cancer-derived Caco-2 cells on a cell culture substrate having an annular cell-adhesive part having an inside diameter of 280 μm and a width of 60 μm in Example 10.

FIG. 24 shows an observation image of a culture on culture day 18 obtained by culturing large intestinal epithelial cancer-derived Caco-2 cells on a cell culture substrate having a semicircular arc-like cell-adhesive part having an inside diameter of 280 μm and a width of 60 μm and lacking ½ in the circumferential direction of a ring in Example 10.

FIG. 25 shows an observation image of a culture on culture day 18 obtained by culturing large intestinal epithelial cancer-derived Caco-2 cells on a cell culture substrate having a square cell-adhesive part having an inside dimension of 280 μm in side and a width of 60 μm in Example 10.

FIG. 26 shows an observation image of a culture on culture day 18 obtained by culturing large intestinal epithelial cancer-derived Caco-2 cells on a cell culture substrate having a C-shaped cell-adhesive part having an inside diameter of 280 μm and a width of 60 μm and lacking ⅛ in the circumferential direction of a ring in Example 10.

FIG. 27 is a schematic view of one embodiment of a cell culture substrate having a cell culture part comprising a non-cell-adhesive part (central part) and a cell-adhesive part surrounding it on each of the upper surfaces of a plurality of protrusions. FIG. 27(A) is a plane view of the cell culture substrate. FIG. 27(B) is a cross-sectional schematic view taken along the A-A line in FIG. 27(A).

FIG. 28 is a schematic view of one embodiment of a cell culture substrate having a cell culture part comprising a non-cell-adhesive part (central part) and a cell-adhesive part surrounding it on each of the bottom surfaces of a plurality of depressions. FIG. 28(A) is a plane view of the cell culture substrate. FIG. 28(B) is a cross-sectional schematic view taken along the A-A line in FIG. 28(A).

FIG. 29 is a schematic view of one embodiment of a cell culture substrate in which a cell culture part comprising a non-cell-adhesive part (central part) and a cell-adhesive part surrounding it occupies the whole of one surface. FIG. 29(A) is a plane view of the cell culture substrate. FIG. 29(B) is a cross-sectional schematic view taken along the A-A line in FIG. 29(A).

### DESCRIPTION OF EMBODIMENTS

### <1. Non-Cell-Adhesive Part and Cell-Adhesive Part of Cell Culture Substrate>

A cell culture substrate according to one or more embodiments of the present disclosure has a surface comprising a cell culture part.

The cell culture part comprises a non-cell-adhesive part, and a cell-adhesive part extending continuously or intermittently along the periphery of the non-cell-adhesive part and surrounding the non-cell-adhesive part.

One or more such cell culture parts are contained on the surface of the cell culture substrate. When two or more cell culture parts are contained, each individual of the cell culture parts may have the features described above.

The non-cell-adhesive part in the cell culture part may also be referred to as a “second non-cell-adhesive part” or a “central part” in order to distinguish it from a non-cell-adhesive part (first non-cell-adhesive part mentioned later) present in a portion other than the cell culture part.

Specifically, the cell culture substrate according to one or more embodiments of the present disclosure has the non-cell-adhesive part and the cell-adhesive part formed on the surface thereof so as to assume predetermined shapes.

Hereinafter, a portion other than the cell culture part in the cell culture substrate may also be referred to as a “support substrate” in order to describe features, other than the cell culture part comprising the non-cell-adhesive part and the cell-adhesive part, of the cell culture substrate according to one or more embodiments of the present disclosure. In other words, the cell culture substrate according to one or more embodiments of the present disclosure comprises a support substrate having a surface comprising the one or more cell culture parts.

Accordingly, embodiments of the support substrate and features other than the shapes of the non-cell-adhesive part and the cell-adhesive part will first be described below.

A support substrate used for a cell culture substrate is not particularly limited as long as the support substrate is formed with a material that allows formation of a non-cell-adhesive part and a cell-adhesive part on the surface thereof. Specific examples of a substrate include: support substrates containing inorganic materials such as glass, a metal, ceramic, and silicon; and organic materials represented by elastomers and plastics (e.g., polystyrene resin, polyester resin, polyethylene resin, polypropylene resin, ABS resin, nylon, acrylic resin, fluororesin, polycarbonate resin, polyurethane resin, methylpentene resin, phenol resin, melamine resin, epoxy resin, and vinyl chloride resin). Particularly, a glass substrate is preferably used as a support substrate. The shape of a support substrate is also not limited, and examples thereof include a flat shape such as a flat substrate, a flat membrane, a film, or a porous membrane, and a three-dimensional shape such as a cylinder, a stamp, a multi-well substrate, or a micro flow path.

According to an embodiment of the present disclosure, the term “cell adhesiveness” means the strength of adhering cells, that is to say, the ease of cell adhesion. The term “cell-adhesive part” refers to a region on the surface with favorable cell adhesiveness, and the term “non-cell-adhesive part” refers to a region on the surface with poor cell adhesiveness. Accordingly, when cells are seeded on the surface on which the cell-adhesive part and the non-cell-adhesive part are arranged with a predetermined pattern, cells adhere to the cell-adhesive parts but not to the non-cell-adhesive part, allowing the cells to be arranged in such pattern on the surface of the cell culture substrate.

The term “cell-adhesive part” is defined as a portion to which cells (preferably stem cells or cancer cells) to be actually cultured adhere when seeded on a cell culture substrate. The term “non-cell-adhesive part” is defined as a portion to which cells (preferably stem cells or cancer cells) to be actually cultured do not adhere when seeded. When cells are seeded on a cell culture substrate, the cell culture substrate may have a surface with enhanced cell adhesiveness by coating with a protein or the like. Specific examples of “stem cells” and “cancer cells” are as mentioned later. The non-cell-adhesive part may be covered with cells that have adhered to the cell-adhesive part and grown.

As an indicator for judging whether to be a cell-adhesive part or to be a non-cell-adhesive part, the cell adhesion/spreading rate when actually culturing cells can be used. The surface of a cell-adhesive part having cell adhesiveness is preferably a surface having a cell adhesion/spreading rate of 60% or more and more preferably a surface having a cell adhesion/spreading rate of 80% or more. When the cell adhesion/spreading rate is high, cells can be cultured efficiently. The cell adhesion/spreading rate according to an embodiment of the present disclosure can be defined as a proportion of adhering/spread cells at a time point when cells to be cultured at a seeding density in a range of 4000 cells/cm² or more to less than 30000 cells/cm² have been seeded on a surface of a measurement subject, stored in an incubator at 37° C. and a CO₂ concentration of 5%, and cultured for 14.5 hours ({(the number of adhering cells)/(the number of seeded cells)}×100(%)).

In the above measurement, cell seeding is conducted in a manner such that cells suspended in a 10% FBS-containing DMEM medium are seeded on a surface to be measured, and then, the surface to be measured on which cells have been seeded is slowly shaken to allow the cells to be distributed as uniformly as possible. Further, the cell adhesion/spreading rate is measured after changing the medium immediately before the measurement to remove non-adhering cells. Upon measurement of the cell adhesion/spreading rate, areas other than areas where the cell presence density is likely to be specific (for example, the center of a predetermined area where the presence density tends to be high and the periphery of a predetermined area where the presence density tends to be low) are determined to be measurement areas.

Meanwhile, the non-cell-adhesive part is a region of a surface having the property that makes cells difficult to adhere (non-cell adhesiveness). Non-cell adhesiveness is determined whether or not cell adhesion or spreading is likely to occur depending on properties such as chemical properties and physical properties of a surface. A surface of the non-cell-adhesive part is a surface having a cell adhesion/spreading rate (defined above) of preferably less than 60%, more preferably less than 40%, further preferably 5% or less, and most preferably 2% or less.

The cell-adhesive part may be a region in which a cell-adhesive layer is formed on the surface of the support substrate. When the surface of the support substrate has cell adhesiveness (e.g., the surface of a glass substrate), the cell-adhesive part may be a region on which the surface of the support substrate is exposed. A region on which the surface having cell adhesiveness of the support substrate is exposed is preferred. The non-cell-adhesive part can be a region in which a non-cell-adhesive layer is formed on the surface of the support substrate. The cell-adhesive part and the non-cell-adhesive part can be formed using various materials and methods. Preferably, the non-cell-adhesive part is a portion in which the surface of the support substrate is coated with a non-cell-adhesive layer such as a layer containing a hydrophilic organic compound such as a hydrophilic polymer. The average thickness of the non-cell-adhesive layer constituting the non-cell-adhesive part is, as described in Patent Literature 4, preferably 0.8 nm to 500 μm, more preferably 0.8 nm to 100 μm, further preferably 1 nm to 10 μm and most preferably 1.5 nm to 1 μm. In a case in which the average thickness is 0.8 nm or more, protein adsorption and cell adhesion are unlikely to be affected by areas other than areas covered with the non-cell-adhesive layer on the support substrate, which is preferable. In addition, in a case in which the average thickness is 500 μm or less, coating is relatively easy. Further, as described in Patent Literature 5, in the case of forming a non-cell-adhesive layer with a layer of polyethylene glycol, one example of its film thickness can include 5 nm to 10 nm. Specific examples of the hydrophilic organic compound are as mentioned later.

A method described in Patent Literature 4 and Non Patent Literature 10 can be used as a method for producing a cell culture substrate containing polyethylene glycol (PEG) as a hydrophilic polymer in a non-cell-adhesive layer.

The following two embodiments are particularly preferable for a method for forming the cell-adhesive part and the non-cell-adhesive part.

In the first embodiment, a non-cell-adhesive layer is formed on the surface of a support substrate. Then, a part of the non-cell-adhesive layer is subjected to a predetermined treatment so that cell adhesiveness is exerted to prepare a cell-adhesive part. Specifically, an exemplary method involves forming a non-cell-adhesive hydrophilic film containing a hydrophilic organic compound such as a hydrophilic polymer, as a non-cell-adhesive layer on the surface of a support substrate, and then selectively subjecting a part of the hydrophilic film serving as a non-cell-adhesive layer to oxidation treatment and/or decomposition treatment to modify the part into a cell-adhesive part having cell adhesiveness. In this embodiment, the cell-adhesive part is prepared by forming a non-cell-adhesive hydrophilic film, and then, applying oxidation treatment and/or decomposition treatment to a site where cell adhesion is desired so as to convert the site to a site having cell adhesiveness. In the cell culture substrate formed according to the first embodiment, the non-cell-adhesive part is a portion in which the surface of the support substrate is coated with a layer containing a hydrophilic organic compound such as a hydrophilic polymer, and the cell-adhesive part is a portion on which the surface of the support substrate is exposed by removing the layer containing a hydrophilic organic compound such as a hydrophilic polymer through oxidation treatment and/or decomposition treatment, or a portion coated with a layer modified so as to have cell adhesiveness (=cell-adhesive layer) from the layer containing a hydrophilic organic compound such as a hydrophilic polymer in response to oxidation treatment and/or decomposition treatment.

In the second embodiment, the cell-adhesive part and the non-cell-adhesive part depend on a high or low density of an organic compound on the surface of a support substrate. In the cell culture substrate formed according to the second embodiment, the cell-adhesive part is a surface having a low density of a hydrophilic organic compound such as a hydrophilic polymer (which also includes the absence of the hydrophilic organic compound), and the non-cell-adhesive part is a surface having a high density of a hydrophilic organic compound such as a hydrophilic polymer. The second embodiment exploits the fact that the surface of the support substrate containing a hydrophilic organic compound such as a hydrophilic polymer at a high density has non-cell-adhesiveness, whereas the surface of the support substrate having a low density of the compound has cell adhesiveness. A first region likely to be bonded to the compound and a second region unlikely to be bonded to the compound are established on a support substrate surface, and a film of the compound is formed on the substrate surface. As a result, the first region becomes a non-cell-adhesive part, and the second region becomes a cell-adhesive region. Alternatively, a part of the support substrate surface is selectively masked with a photoresist, and a film of the hydrophilic organic compound is formed on an unmasked region to form a non-cell-adhesive part. Then, the masking is removed so that the surface of the support substrate can be exposed to form a cell-adhesive part.

Instead of these embodiments, a support substrate having a non-cell-adhesive surface (which may be a surface of a non-cell-adhesive layer) is prepared, and a part of the surface may be coated by patterning with a cell-adhesive protein such as collagen or fibronectin to form a cell-adhesive pattern. Alternatively, a support substrate having a cell-adhesive surface (which may be a surface of a cell-adhesive layer) is prepared, and a part of the surface may be coated with a non-cell-adhesive resin such as silicone rubber (e.g., KEIJU® manufactured by Mitsubishi Chemical Corp.) to prepare a cell adhesive pattern as a remnant. Alternatively, a support substrate provided with a conductive layer having a predetermined pattern on the surface is prepared, and the surface of the support substrate is covered with a non-cell-adhesive layer. The coating with the non-cell-adhesive layer on the conductive layer is detached by voltage application to the conductive layer, and the conductive layer thus exposed may be used as a cell-adhesive part (for the details, see JP Patent Publication (Kokai) No. 2012-120443 A (2012) and JP Patent Publication (Kokai) No. 2013-179910 A (2013)).

Hereinafter, the first and second embodiments will be described in order in which a cell-adhesive part and a non-cell-adhesive part are formed on a support substrate surface to produce a cell culture substrate having a surface comprising the cell-adhesive part and the non-cell-adhesive part.

First, the first embodiment will be described.

In the first embodiment, a hydrophilic film containing a hydrophilic organic compound, preferably a hydrophilic polymer, is first established as a non-cell-adhesive layer on a support substrate surface. The hydrophilic film is a thin film having water solubility or water swellability, and is not particularly limited as long as it has non-cell-adhesiveness before being oxidated and/or decomposed and an exposed surface of the support substrate after oxidation and/or decomposition or the surface of the thin film modified in response to oxidation treatment and/or decomposition treatment exhibits cell adhesiveness.

When the non-cell-adhesive layer is a hydrophilic film formed from a hydrophilic organic compound, it is preferable to establish a bonding layer, if necessary, between the surface of the support substrate and the hydrophilic film. The bonding layer is preferably a layer containing a material having a functional group capable of being bonded (bonding functional group) to a functional group of the organic compound in the hydrophilic film. Examples of a combination of the bonding functional group of the material of the bonding layer and the functional group of the hydrophilic organic compound include an epoxy group and a hydroxy group, phthalic anhydride and a hydroxy group, a carboxyl group and N-hydroxysuccinimide, a carboxyl group and carbodiimide, and an amino group and glutaraldehyde. Either of the functional groups in each combination may be present in the bonding layer. In these methods, a bonding layer is formed with a material having a predetermined functional group on a support substrate before coating with a hydrophilic organic compound. In the non-cell-adhesive layer, in one example of a silane coupling agent having a terminal epoxy group for use as a material having a bonding functional group, the water contact angle on the surface of the bonding layer before formation of a thin film of a hydrophilic organic compound is typically 45° or more, desirably 47° or more. Such a bonding layer is obtained by forming a coating of a material having a bonding functional group on the surface of a support substrate.

Examples of the hydrophilic organic compound include hydrophilic polymers (including hydrophilic oligomers), water-soluble organic compounds, surface active materials, and amphipathic materials. A hydrophilic polymer is particularly preferable.

Specific examples of the hydrophilic polymer can include polyalkylene glycol, zwitterionic polymers having a phospholipid polar group, polyacrylamide, polyacrylic acid, polymethacrylic acid, polyvinyl alcohol, and polysaccharides. Specific examples of these hydrophilic polymers also include their derivative forms. Examples of the molecular shape of the hydrophilic polymer can include linear or branched polymers and dendrimers.

Specifically, the polyalkylene glycol is preferably polyethylene glycol, polypropylene glycol, or a copolymer of polyethylene glycol and polypropylene glycol, for example, Pluronic F108 or Pluronic F127.

Specifically, the zwitterionic polymer having a phospholipid polar group is preferably poly(methacryloyloxyethyl phosphorylcholine) (=MPC polymer), a copolymer of methacryloyloxyethyl phosphorylcholine and an acrylic monomer, or the like.

Specific examples of the polyacrylamide can include poly(N-isopropylacrylamide).

Specific examples of the polymethacrylic acid can include poly(2-hydroxyethyl methacrylate).

Specific examples of the polysaccharide can include dextran and heparin.

It is desirable that the surface of the support substrate having a non-cell-adhesive layer should have high non-cell-adhesiveness in a state coated with the non-cell-adhesive layer and an exposed surface of the support substrate after oxidation treatment and/or decomposition treatment of the non-cell-adhesive layer, or the surface of a layer formed by modifying the non-cell-adhesive layer through oxidation treatment and/or decomposition treatment should exhibit cell adhesiveness.

The hydrophilic polymer is particularly preferably polyethylene glycol (PEG). PEG includes at least an ethylene glycol chain (EG chain) comprising at least one ethylene glycol unit ((CH₂)₂—O), which may be either linear or branched chain. The ethylene glycol chain has a structure expressed by, for example, the following formula:

—((CH₂)₂—O)m-

(m is an integer indicating the degree of polymerization).
m is preferably an integer of 1 to 13 and more preferably an integer of 1 to 10.

PEG also includes ethylene glycol oligomer. In addition, PEG may be functional-group-containing PEG. Examples of a functional group include an epoxy group, a carboxyl group, an N-hydroxysuccinimide group, a carbodiimide group, an amino group, a glutaraldehyde group, and a (meth)acryloyl group. A functional group is optionally introduced via a linker, preferably at the end of the chain. Examples of functional-group-containing PEG include PEG(meth)acrylate and PEG di(meth)acrylate.

The cell-adhesive part can be formed by applying oxidation treatment and/or decomposition treatment to a non-cell-adhesive layer containing a hydrophilic organic compound formed on the surface of a support substrate so that the surface of the support substrate having cell adhesiveness is exposed or so that the non-cell-adhesive layer is modified and converted into a cell-adhesive layer.

According to an embodiment of the present disclosure, the word “oxidation” has a narrow meaning and refers to a reaction in which an organic compound reacts with oxygen and the content of oxygen becomes larger than that before the reaction.

According to an embodiment of the present disclosure, the word “decomposition” refers to a change in which a bond of an organic compound is cleaved to generate two or more organic compounds from the organic compound. Typically, “decomposition treatment” is caused by, but is not limited to, oxidation treatment, ultraviolet irradiation, or other treatments. In a case in which “decomposition treatment” is accompanied by oxidation (i.e., oxidation decomposition), “decomposition treatment” and “oxidation treatment” refer to the same treatment. The removal of a non-cell-adhesive layer by decomposition is also included in “decomposition treatment”.

Decomposition by ultraviolet irradiation means that an organic compound absorbs ultraviolet rays and decomposes via an excited state. When a system in which an organic compound is present together with molecular species containing oxygen (e.g., oxygen or water) with ultraviolet rays, ultraviolet rays are absorbed by the organic compound and decomposition occurs, and in some cases, the molecular species is activated to react with the organic compound. The latter reaction can be classified as “oxidation.” A reaction in which an organic compound is decomposed via oxidation caused by the activated molecular species can be classified as “decomposition by oxidation” rather than “decomposition by ultraviolet irradiation.”

As described above, “oxidation treatment” and “decomposition treatment” may be duplicated as operations, and therefore, they cannot be distinguished clearly. In view of this, the term “oxidation treatment and/or decomposition treatment” is used herein.

Next, the second embodiment will be described. In the cell culture substrate formed according to the second embodiment, the cell-adhesive part in the surface of the support substrate is a surface having a low density of a hydrophilic organic compound such as a hydrophilic polymer (which also includes the absence of the hydrophilic organic compound), and the non-cell-adhesive part is a surface having a high density of a hydrophilic organic compound. In other words, the cell-adhesive part and the non-cell-adhesive part differ in the density of a hydrophilic organic compound. A higher density thereof tends to render cells more unlikely to adhere. In the cell-adhesive part, the density of the hydrophilic organic compound is low to an extent that cells can adhere thereto. Preferred examples of the hydrophilic organic compound and the hydrophilic polymer are as already mentioned about the first embodiment.

In the second embodiment, in the case of forming a cell-adhesive part and a non-cell-adhesive part with a hydrophilic film having a controlled density, it is preferable to form a bonding layer, if necessary, on a support substrate in order to enhance adhesion to the support substrate, and then form a hydrophilic film made of a hydrophilic organic compound. The bonding layer is preferably a layer containing a material containing a functional group capable of being bonded (bonding functional group) to a functional group of the hydrophilic organic compound. Examples of a combination of the functional group of the material of the bonding layer and the functional group of the hydrophilic organic compound include an epoxy group and a hydroxy group, phthalic anhydride and a hydroxy group, a carboxyl group and N-hydroxysuccinimide, a carboxyl group and carbodiimide, and an amino group and glutaraldehyde. Either of the functional groups in each combination may be present in the bonding layer. In these methods, a bonding layer is formed with a material having a predetermined functional group on a support substrate before coating with a hydrophilic material. The density of the material in the bonding layer is an important factor for defining a bonding force. The density can be readily evaluated based on the water contact angle as an index on the surface of the bonding layer. The water contact angle is a value measured 30 seconds after dropwise addition of pure water from a microsyringe using CA-Z manufactured by Kyowa Interface Science Co., Ltd.

In the bonding layer of the cell-adhesive part, the material having a bonding functional group has a low density. In the cell-adhesive part, in an example of a silane coupling agent having a terminal epoxy group for use as a material having a bonding functional group constituting the bonding layer, the water contact angle on the surface of the bonding layer before formation of a thin film of a hydrophilic organic compound is typically 10° to 43°, desirably 15° to 40°. Examples of a method for forming such a bonding layer include a method of forming a coating of a material having a bonding functional group (bonding layer) on the surface of a support substrate, followed by oxidation treatment and/or decomposition treatment of the surface of the bonding layer. Examples of an oxidation treatment and/or decomposition treatment method for a bonding layer surface include a method for irradiating a bonding layer surface with ultraviolet rays, a method for treating a bonding layer surface with a photocatalyst, and a method for treating a bonding layer surface with an oxidizing agent. The whole bonding layer surface may be subjected to oxidation treatment and/or decomposition treatment, or the bonding layer surface may be partially treated. The partial treatment can be performed by using a mask such as a photomask or a stencil mask or using a stamp. In addition, oxidation treatment and/or decomposition treatment may be carried out by a direct drawing method such as a method using laser such as ultraviolet laser. As for conditions, etc., the same conditions as in a method for forming a cell-adhesive part by the oxidation treatment and/or decomposition treatment of a hydrophilic film can be applied. The cell-adhesive part can be formed by forming a thin film of a hydrophilic organic compound on the bonding layer thus formed.

In the bonding layer of the non-cell-adhesive part, the material having a bonding functional group has a high density. In the non-cell-adhesive part, in an example of a silane coupling agent having a terminal epoxy group for use as a material having a bonding functional group, the water contact angle on the surface of the bonding layer before formation of a thin film of a hydrophilic organic compound is typically 45° or more, desirably 47° or more. Such a bonding layer is obtained by forming a coating of a material having a bonding functional group on the surface of a support substrate. In the case of partially subjecting a bonding layer surface to oxidation treatment and/or decomposition treatment, a residual portion that has not undergone the treatment becomes a bonding layer having the water contact angle. The non-cell-adhesive layer can be formed by forming a thin film of a hydrophilic organic compound on the bonding layer thus formed.

In the second embodiment, a part of the support substrate surface is selectively masked with a photosensitive photoresist, and a film of the hydrophilic organic compound is formed on an unmasked region to form a non-cell-adhesive part. Then, the masking is removed so that the surface of the support substrate can be exposed to form a cell-adhesive part.

Subsequently, features of a cell-adhesive part and a non-cell-adhesive part formed according to the first or second embodiment described above or other methods will be further described.

The amount of carbon in a cell-adhesive part (including a bonding layer when there is a bonding layer) is preferably lower than the amount of carbon in a non-cell-adhesive part (including a bonding layer when there is a bonding layer). Specifically, the amount of carbon in a cell-adhesive part is preferably 20% to 99% of the amount of carbon in a non-cell-adhesive part. This range is appropriate, particularly, when the thickness of a hydrophilic organic compound layer (the total thickness of a bonding layer and the hydrophilic film when there is a bonding layer) contained in a cell-adhesive part or a non-cell-adhesive part is 10 μm or less. “Amount of carbon (atomic concentration, %)” is as defined below.

In addition, the proportion of carbon bonded to oxygen (%) with respect to carbon present in a cell-adhesive part (including a bonding layer when there is a bonding layer) is preferably smaller than the proportion of carbon bonded to oxygen (%) with respect to carbon present in a non-cell-adhesive part (including a bonding layer when there is a bonding layer). Specifically, the proportion of carbon bonded to oxygen (%) with respect to carbon present in a cell-adhesive part is preferably 35% to 99% of the proportion of carbon bonded to oxygen (%) with respect to carbon present in a non-cell-adhesive part. This range is appropriate, particularly, when the thickness of a hydrophilic film (the total thickness of a bonding layer and the hydrophilic film when there is a bonding layer) is 10 μm or less. “Proportion of carbon bonded to oxygen (atomic concentration, %)” is as defined below.

Contact angle measurement, ellipsometry, atomic force microscopy, electron microscopy, Auger electron spectroscopy, X-ray photoelectron spectroscopy, various mass spectrometry methods, or the like can be used as an approach of evaluating a hydrophilic organic compound layer (including a bonding layer when there is a bonding layer) contained in a cell-adhesive part or a non-cell-adhesive part. Among these approaches, X-ray photoelectron spectroscopy (XPS/ESCA) has the best quantitativeness. A relative quantitative value is determined by this measurement method and is generally calculated in atomic concentration (%). Hereinafter, an X-ray photoelectron spectroscopy method according to an embodiment of the present disclosure will be described in detail.

“The amount of carbon” in a cell-adhesive part and a non-cell-adhesive part is defined as “the amount of carbon calculated from the analysis value of the C1s peak obtained by using an X-ray photoelectron spectrometer”. According to an embodiment of the present disclosure, “the proportion of carbon bonded to oxygen” in a cell-adhesive part and a non-cell-adhesive part is defined as “the proportion of carbon bonded to oxygen calculated from the analysis value of the C1s peak obtained by using an X-ray photoelectron spectrometer.” Specific measurement can be carried out as described in JP Patent Publication (Kokai) No. 2007-312736 A (2007).

### <2. Feature of Shape of Cell Culture Part in Cell Culture Substrate>

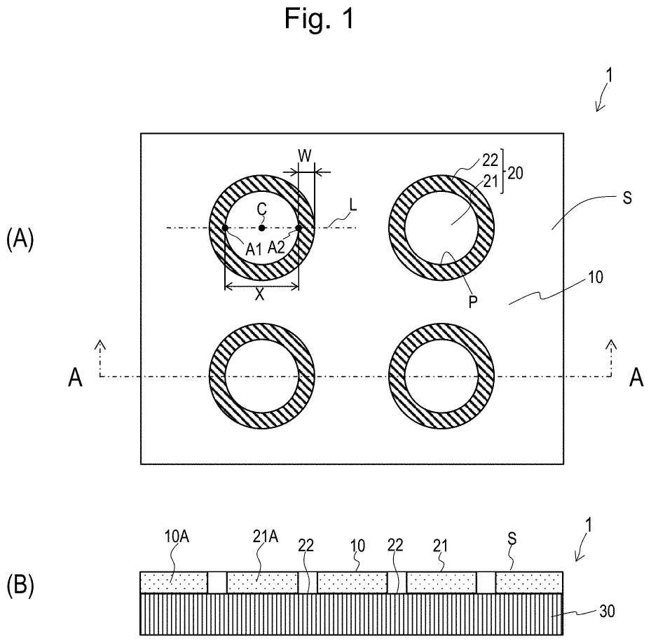

Features of a cell culture substrate used in an embodiment of the present disclosure will be described, mainly, with reference to FIG. 1.

Cell culture substrate 1 used in an embodiment of the present disclosure has surface S comprising cell culture part 20.

The cell culture part 20 has non-cell-adhesive part (central part) 21, and cell-adhesive part 22 extending continuously or intermittently (FIG. 1 shows an example of extending continuously) along periphery P of the non-cell-adhesive part 21 and surrounding the non-cell-adhesive part 21. This embodiment in an example in which one or more cell culture parts 20 are contained on surface S of cell culture substrate 1 and each of the one or more cell culture parts 20 has the features described above.

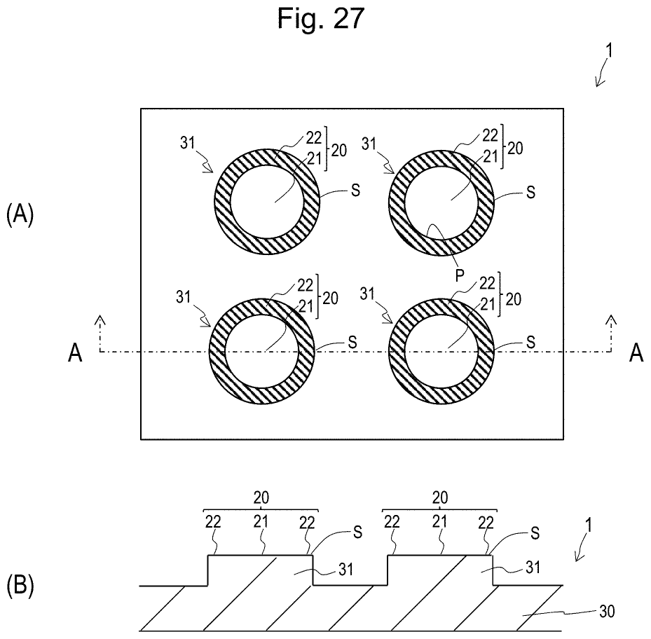

In the example shown in FIG. 1, one or more cell culture parts 20 are scattered in the form of islands in non-cell-adhesive part 10. In this example, the non-cell-adhesive part 10 may also be referred to as “first non-cell-adhesive part”, and the non-cell-adhesive part 21 in the cell-adhesive part 20 may also be referred to as “second non-cell-adhesive part”. In the description below, the “non-cell-adhesive part 21” may also be referred to as “central part 21” or “central part 21 serving as a non-cell-adhesive part”. The first non-cell-adhesive part 10 is not an essential constituent. An example of a cell culture substrate having no first non-cell-adhesive part 10 will be separately described with reference to FIGS. 27 to 29.

In the cell culture substrate 1, a portion in which first non-cell-adhesive part 10 and cell-adhesive part 20 are arranged on the surface is referred to as “support substrate 30”.

In the example shown in FIG. 1, the first non-cell-adhesive part 10 and the central part 21 serving as a second non-cell-adhesive part are the surfaces of first non-cell-adhesive layer 10A and second non-cell-adhesive layer 21A, respectively, disposed on the surface of the support substrate 30.

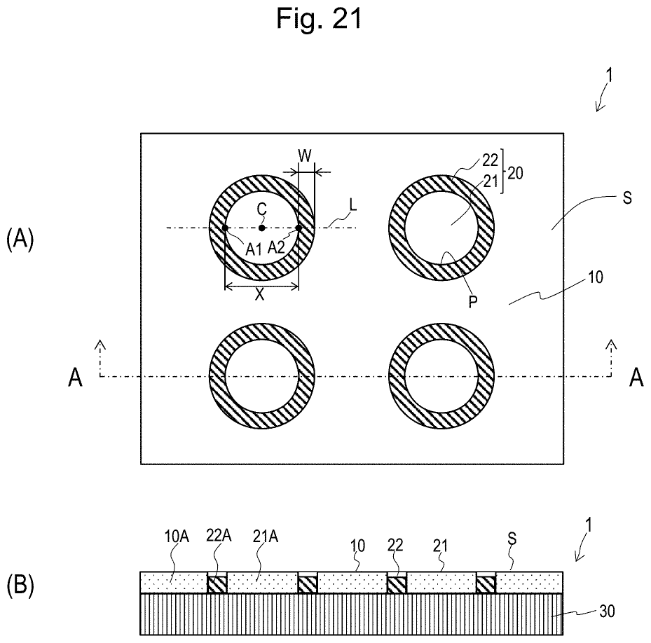

In the example shown in FIG. 1, the cell-adhesive part 22 is an exposed surface of the support substrate 30. As in an example shown in FIG. 21, the cell-adhesive part 22 may be the surface of cell-adhesive layer 22A disposed on the surface of the support substrate 30.

In FIG. 1(B) and FIG. 21(B), the thicknesses of the non-cell-adhesive layer 10A, the non-cell-adhesive layer 21A and the cell-adhesive layer 22A as well as the level difference between the cell-adhesive part 22 and the non-cell-adhesive layer 10A or the non-cell-adhesive layer 21A is emphatically shown for the sake of convenience. However, the thickness and the level difference are sufficiently small with respect to the sizes of cells to be cultured and the cell construct. Therefore, surface S comprising one or more cell culture parts 20 can support cells as a substantially flat surface.

FIG. 1(B) shows an example in which the support substrate 30 is in direct contact with the first non-cell-adhesive layer 10A and the second non-cell-adhesive layer 21A. As already mentioned, a bonding layer may intervene therebetween. Likewise, FIG. 21(B) shows an example in which the support substrate 30 is in direct contact with the first non-cell-adhesive layer 10A, the second non-cell-adhesive layer 21A, and the cell-adhesive layer 22A. As already mentioned, a bonding layer may intervene therebetween.

Specific examples and production methods of the support substrate 30, the first non-cell-adhesive part 10, the first non-cell-adhesive layer 10A, the second cell-adhesive part 21, the second non-cell-adhesive layer 21A, the cell-adhesive part 22, and the cell-adhesive layer 22A are as already mentioned.

The present inventors have found that, surprisingly, when cells are cultured on cell culture substrate 1 having such a structure, cells attach and grow within cell-adhesive part 22 surrounding non-cell-adhesive part (central part) 21 to form an accumulating portion having dense accumulations of cells and a sac-shaped cell construct (tissue) is easily formed. The present inventors have found that the culture of cells using a cell culture substrate having the structure allows a sac-shaped cell construct to be released from the cell culture substrate in a relatively short time and collected and furthermore, collection efficiency is exceedingly high. Examples of cells that can form a sac-shaped cell construct can include stem cells and cancer cells. For example, when stem cells are cultured on cell culture substrate 1, stem cells attach and grow within cell-adhesive part 22 surrounding non-cell-adhesive part (central part) 21 to form an accumulating portion. In the accumulating portion, the stem cells are induced to differentiate into intestinal epithelial cells expressing a trophectodermal cell marker. The obtained sac-shaped cell construct comprises small intestinal epithelial cells and has functions as a gut organoid.

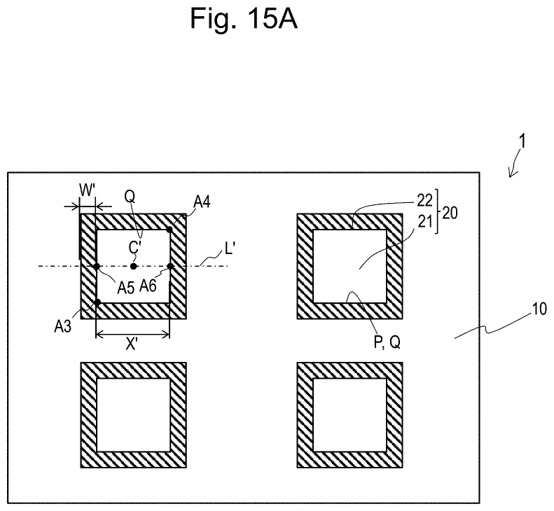

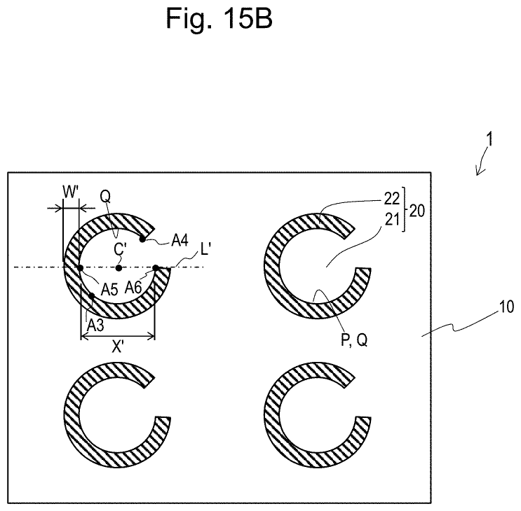

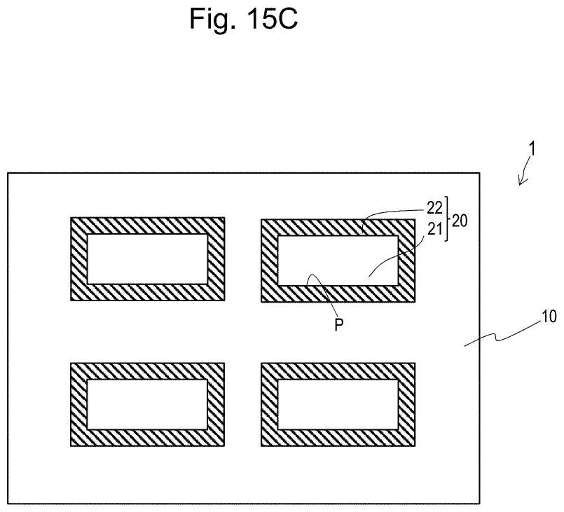

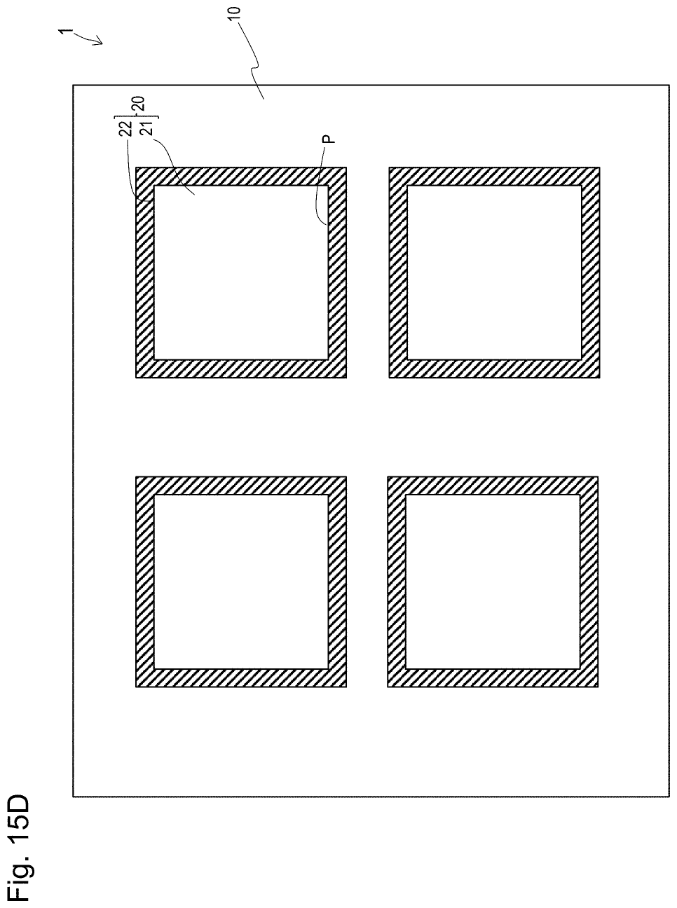

The shape or size of the cell culture part 20 is not particularly limited. In a preferable embodiment, distance X between two points of intersection, A1 and A2, of periphery P of the central part 21 with straight line L passing through barycenter C of the central part 21 is larger than 80 μm and 880 μm or smaller, more preferably 180 μm or larger and 880 μm or smaller, particularly preferably 180 μm or larger and 600 μm or smaller, and particularly preferably 180 μm or larger and 500 μm or smaller. If the distance X is too small, a specific sac-shaped structure is unlikely to be obtained on the outer circumference of the cell construct because the central part 21 is immediately covered with cells during growing culture. On the other hand, if the distance X is too large, the production efficiency of the cell construct is reduced because the time required for cells to grow and completely cover the central part 21 is long. When the distance X falls within the above range, a cell construct having a sac-shaped structure can be cultured at high yields in a relatively short time. The distance X refers to the diameter of a circle when the shape of the central part 21 is a circle as shown in FIG. 1 and FIG. 15B. When the circle is a true circle, the distance X is constant regardless of how the straight line L is drawn. When the central part 21 is rectangular as shown in FIG. 15A, 15C or 15D, the distance X is largest with the straight line L drawn in the diagonal direction and is smallest with the straight line L drawn in the shorter direction. According to an embodiment of the present disclosure, preferably, the distance X falls within the above range over the whole circumference (i.e., for any straight lines L drawn).

In another preferable embodiment of the cell culture part 20, width W of the cell-adhesive part 22 along the straight line L passing through barycenter C of the central part 21 is larger than 30 μm and 400 μm or smaller, more preferably 40 μm or larger and 400 μm or smaller, and particularly preferably 60 μm or larger and 300 μm or smaller. If the width W is too small, cells are disadvantageously likely to be detached during culture. For the induction of a sac-shaped cell construct, it is desirable that a plurality of cells should adhere in the width direction of the cell-adhesive part 22 to form an accumulating portion. For this purpose, larger width W is preferable. Therefore, as described above, the width W is preferably 40 μm or larger, more preferably 60 μm or larger. On the other hand, if the width W is too large, a cell construct having a uniform structure is unlikely to be obtained because the density of cells adhering to the cell-adhesive part 22 is likely to be biased and makes it difficult to form an accumulating portion of cells uniform in the width direction. When the width W falls within the above range, a cell construct can be cultured at high yields in a relatively short time. The width W refers to the width of the cell-adhesive part 22 in the diameter direction of a circle when the shape of the central part 21 is a circle as shown in FIG. 1 and FIG. 15B. When the circle is a true circle, the width W is constant regardless of how the straight line L is drawn. When the central part 21 is rectangular as shown in FIG. 15A, 15C or 15D, the width W is largest with the straight line L drawn in the diagonal direction and is smallest with the straight line L drawn in the shorter direction. According to an embodiment of the present disclosure, preferably, the width W falls within the above range over the whole circumference (i.e., for any straight lines L drawn).

In an alternative preferable embodiment of the cell culture part 20, ratio X/W of distance X to width W is preferably 0.5 or more, more preferably 1.0 or more, and further preferably 1.3 or more, and is preferably 20.0 or less, more preferably 15.0 or less, and further preferably 10.0 or less. The distance X is a distance between two points of intersection, A1 and A2, of periphery P of the central part 21 with straight line L passing through barycenter C of the central part 21. The width W is a width of the cell-adhesive part 22 along the straight line L. When the ratio X/W falls within the above range, a cell construct can be cultured at high yields in a relatively short time. According to an embodiment of the present disclosure, preferably, the ratio X/W falls within the above range over the whole circumference (i.e., for any straight lines L drawn).

The shape and size of the cell-adhesive part 22 can be defined by midpoint C′, distance X′, width W′, and straight line L′ described below instead of barycenter C, distance X, width W, and straight line L described above. The midpoint C′, the distance X′, the width W′, and the straight line L′ will be described with reference to FIG. 15A and FIG. 15B. A straight line passing through midpoint C′ between two points, A3 and A4, on an inner circumference Q of the cell-adhesive part 22, opposed to and most distant from each other across the central part 21 is defined as straight line L′. A distance between two points of intersection, A5 and A6, of this straight line L′ with the inner circumference Q of the cell-adhesive part 22 is defined as distance X′. A width of the cell-adhesive part 22 along the straight line L′ passing through midpoint C′ is defined as width W′.

The distance X′ is preferably larger than 80 μm and 880 μm or smaller, more preferably 180 μm or larger and 880 μm or smaller, particularly preferably 180 μm or larger and 600 μm or smaller, and particularly preferably 180 μm or larger and 500 μm or smaller. If the distance X′ is too small, a specific sac-shaped structure is unlikely to be obtained on the outer circumference of the cell construct because the central part 21 is immediately covered with cells during growing culture. On the other hand, if the distance X′ is too large, the production efficiency of the cell construct is reduced because the time required for cells to grow and completely cover the central part 21 is long. When the distance X′ falls within the above range, a cell construct having a sac-shaped structure can be cultured at high yields in a relatively short time. The distance X′ refers to the diameter of a circle when the shape of the central part 21 is a circle as shown in FIG. 1 and FIG. 15B. When the circle is a true circle, the distance X′ is constant regardless of how the straight line L′ is drawn. When the central part 21 is rectangular as shown in FIG. 15A, 15C or 15D, the distance X′ is largest with the straight line L′ drawn in the diagonal direction and is smallest with the straight line L′ drawn in the shorter direction. According to an embodiment of the present disclosure, preferably, the distance X′ falls within the above range over the whole circumference (i.e., for any straight lines L′ drawn).

The width W′ is preferably larger than 30 μm and 400 μm or smaller, more preferably 40 μm or larger and 400 μm or smaller, and particularly preferably 60 μm or larger and 300 μm or smaller. If the width W′ is too small, cells are disadvantageously likely to be detached during culture. For the induction of a sac-shaped cell construct, it is desirable that a plurality of cells should adhere in the width direction of the cell-adhesive part 22 to form an accumulating portion. For this purpose, larger width W′ is preferable. Therefore, as described above, the width W′ is preferably 40 μm or larger, more preferably 60 μm or larger. On the other hand, if the width W′ is too large, a cell construct having a uniform structure is unlikely to be obtained because the density of cells adhering to the cell-adhesive part 22 is likely to be biased and makes it difficult to form an accumulating portion of cells uniform in the width direction. When the width W′ falls within the above range, a cell construct can be cultured at high yields in a relatively short time. The width W′ refers to the width of the cell-adhesive part 22 in the diameter direction of a circle when the shape of the central part 21 is a circle as shown in FIG. 1 and FIG. 15B. When the circle is a true circle, the width W′ is constant regardless of how the straight line L′ is drawn. When the central part 21 is rectangular as shown in FIG. 15A, 15C or 15D, the width W′ is largest with the straight line L′ drawn in the diagonal direction and is smallest with the straight line L′ drawn in the shorter direction. According to an embodiment of the present disclosure, preferably, the width W′ falls within the above range over the whole circumference (i.e., for any straight lines L′ drawn).

Ratio X′/W′ is preferably 0.5 or more, more preferably 1.0 or more, and further preferably 1.3 or more, and is preferably 20.0 or less, more preferably 15.0 or less, and further preferably 10.0 or less. When the ratio X′/W′ falls within the above range, a cell construct can be cultured at high yields in a relatively short time. According to an embodiment of the present disclosure, preferably, the ratio X′/W′ falls within the above range over the whole circumference (i.e., for any straight lines L′ drawn).

In FIG. 1 and FIG. 15B, the central part 21 is round, and the cell-adhesive part 22 has an annular shape concentrically surrounding the round central part 21. This high symmetric fashion is particularly preferable for obtaining a uniform cell construct. However, the present invention is not limited by such an example, and an inner outline and an outer outline may have a rectangular shape, as shown in FIG. 15A, FIG. 15C, or FIG. 15D, in which the central part 21 is rectangular (square or rectangle) and the cell-adhesive part 22 is disposed along periphery P of the rectangular central part 21. Although not shown, the central part may be oval, and the cell-adhesive part may have an oval annular shape extending along the central part. In the examples described above, the inner outline and the outer outline of the cell-adhesive part have similar shapes, though not limited thereto. For example, the inner outline of the cell-adhesive part (i.e., the outer outline of the central part) may be polygonal (e.g., rectangular), and the outer outline of the cell-adhesive part may be round or oval; or alternatively, the inner outline of the cell-adhesive part (i.e., the outer outline of the central part) may be round or oval, and the outer outline of the cell-adhesive part may be polygonal (e.g., rectangular). Alternatively, the central part 21 may be semicircular.

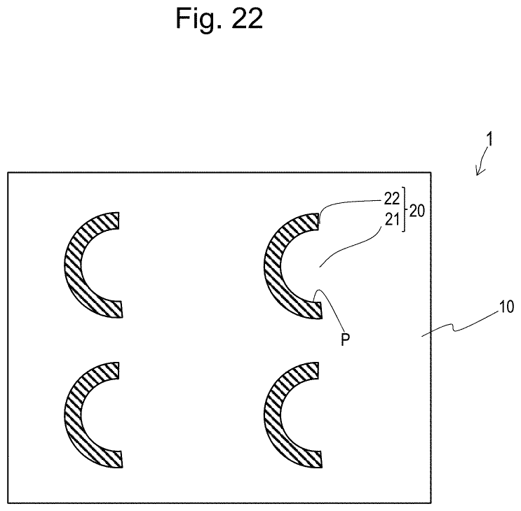

In the examples of FIG. 1, FIG. 15A, FIG. 15C, and FIG. 15D, the cell-adhesive part 22 extends continuously along periphery P of the central part 21 serving as a non-cell-adhesive part and surrounds the central part 21 over the whole circumference. However, the cell-adhesive part may have a shape extending intermittently. Specifically, as shown in the example of FIG. 15B and FIG. 22, the cell-adhesive part 22 extends intermittently along periphery P of the central part 21 serving as a non-cell-adhesive part and surrounds the central part 21. Even in such a structure, cells that have adhered on the cell-adhesive part 22 can form a tissue through growth so as to connect the gap portion of the cell-adhesive part 22. In an embodiment in which the cell-adhesive part 22 extends intermittently along periphery P of the central part 21, the discontinued portion has a length of preferably ½ or less, more preferably ¼ or less, further preferably ⅙ or less, and furthermore preferably ⅛ or less, of the complete periphery P of the central part 21 per location. In the case of including a plurality of discontinued portions, the discontinued portions have a total length of preferably ½ or less, more preferably ¼ or less, further preferably ⅙ or less, and furthermore preferably ⅛ or less, of the complete periphery P of the central part 21.

A cell culture substrate used in an embodiment of the present disclosure has a structure where the cell-adhesive part extends so as to surround the non-cell-adhesive central part, whereby cells that attach and grow on this cell-adhesive part become dense and are more likely to induce a sac-shaped cell construct.

Particularly, in the case of culturing stem cells on a cell culture substrate according to an embodiment of the present disclosure, stem cells that attach and grow on the cell-adhesive part become dense and are more likely to differentiate into cells having the properties of trophectodermal cells while grown cells are likely to accumulate. As a result, a sac-shaped cell construct in which cells having the properties of trophectodermal cells are distributed on the outer circumference can be efficiently obtained.

By contrast, in an approach described in Patent Literature 2 and Non Patent Literature 3, a cell-adhesive part has a round shape which causes seeded cells to spread into the inside through growth. Thus, a sac-shaped cell construct is difficult to form because cells are unlikely to accumulate in the outer circumference.

Particularly, in the case of culturing stem cells on a cell culture substrate having a round cell-adhesive part described in Patent Literature 2 and Non Patent Literature 3, cells having the properties of trophectodermal cells spread into the inside through growth. Thus, a sac-shaped cell construct in which cells having the properties of trophectodermal cells are distributed on the outer circumference is unlikely to be obtained and is also unlikely to be induced to differentiate into endodermal cells. Hence, it is considered that a collection rate was decreased as shown in results of Comparative Example 1 mentioned later. When a cell-adhesive part has a round shape, it is considered that the cell-adhesive part had a large area which consumed a time for forming an accumulating portion.

A plurality of cell culture parts 20, if present, as in cell culture substrate 1 are isolated from each other and arranged apart from each other with a distance of preferably 0.20 mm or more and more preferably 0.30 mm or more. By arranging cell culture parts 20 to be apart from each other with not less than a certain distance, cells within each cell culture part 20 are uniformly cultured with a certain distance from each of other cell culture parts 20 without forming intercellular bonds with cells in other cell culture parts 20 adjacent thereto, thereby making it possible to construct an experimental system with high reproducibility.

Cell culture substrate 1 according to each of the embodiments shown in FIG. 1, FIG. 15A, FIG. 15B, FIG. 15C, FIG. 15D, and FIG. 21 has a structure where one or more cell culture parts 20 are present in first non-cell-adhesive part 10. An embodiment of a cell culture substrate having no first non-cell-adhesive part 10 will be separately described with reference to FIGS. 27 to 29.

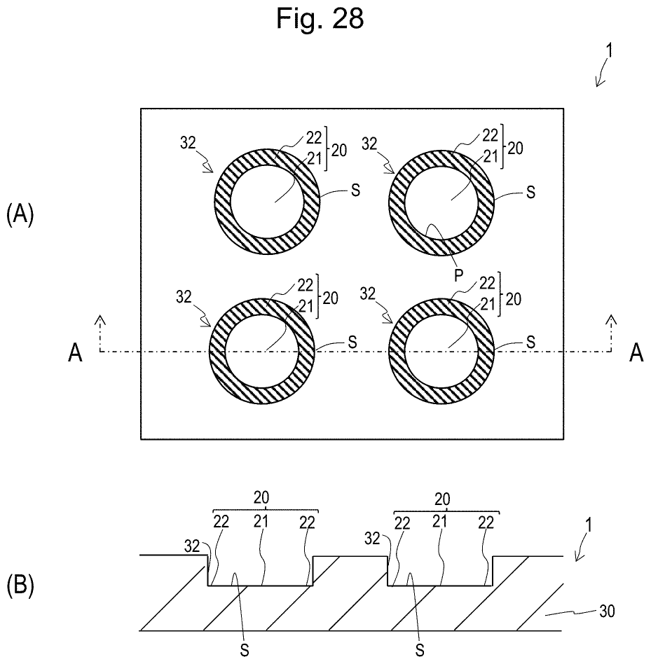

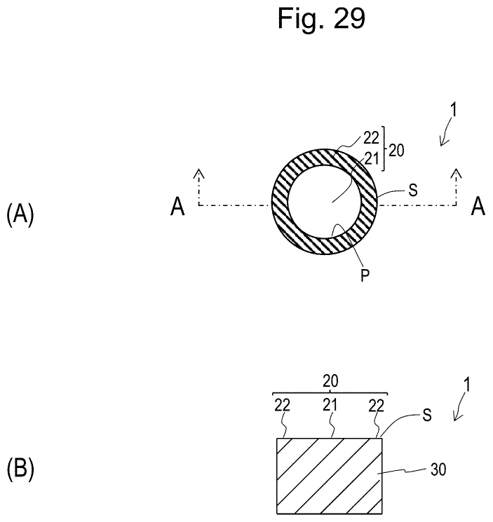

Differences of cell culture substrate 1 shown in each of FIGS. 27 to 29 from cell culture substrate 1 shown in FIG. 1 or FIG. 21 will be described below. In cell culture substrate 1 shown in each of FIGS. 27 to 29, features and formation methods of non-cell-adhesive part (central part) 21 and cell-adhesive part 22 constituting cell culture part 20 are the same as in non-cell-adhesive part 21 and cell-adhesive part 22 in cell culture substrate 1 shown in FIG. 1 or FIG. 21, so that the description about cross-sectional features of non-cell-adhesive part 21 and cell-adhesive part 22 on the cross section of cell culture substrate 1 in FIG. 27(B), FIG. 28(B) or FIG. 29(B) is omitted. Other features that are not mentioned about cell culture substrate 1 shown in each of FIGS. 27 to 29 are the same as in cell culture substrate 1 shown in FIG. 1 or FIG. 21, so that the description is omitted.

Cell culture substrate 1 according to one embodiment of the present disclosure shown in FIG. 27 has surface S comprising cell culture part 20. The cell culture part 20 has non-cell-adhesive part 21, and cell-adhesive part 22 extending continuously or intermittently (FIG. 27 shows an example of extending continuously) along periphery P of the non-cell-adhesive part 21 and surrounding the non-cell-adhesive part 21. In the cell culture substrate 1 shown in FIG. 27, a portion the surface on which cell-adhesive part 20 is arranged is referred to as “support substrate 30”.

The cell culture substrate 1 shown in FIG. 27 has support substrate 30 having one or more protrusions 31, and comprises non-cell-adhesive part 21 and cell-adhesive part 22 surrounding the non-cell-adhesive part 21 on upper surface S of each protrusion 31. In this embodiment, the protrusion 31 has round upper surface S, though the protrusion 31 may have other shapes. In this embodiment, the cell-adhesive part 22 is present on the periphery of the upper surface S of the protrusion 31, and the support substrate is absent outside the cell-adhesive part 22, in a planar view. Therefore, cells that have adhered to the cell-adhesive part 22, when cultured, do not spread to the outside of the cell-adhesive part 22, and spread on the cell-adhesive part 22 and the non-cell-adhesive part 21 thereinside to form a cell construct.

According to the embodiment shown in FIG. 27, since cell culture parts 20 (each consisting of non-cell-adhesive part 21 and cell-adhesive part 22) are present on upper surfaces S of protrusions 31 isolated from each other in the horizontal direction, cells within each cell culture part 20 are cultured without forming intercellular bonds with cells in other cell culture parts 20 adjacent thereto, easily constructing an experimental system with high reproducibility. In this embodiment, a plurality of protrusions 31, if present, are isolated from each other and arranged apart from each other with a distance of preferably 0.20 mm or more and more preferably 0.30 mm or more.

Cell culture substrate 1 according to one embodiment of the present disclosure shown in FIG. 28 has surface S comprising cell culture part 20. The cell culture part 20 has non-cell-adhesive part 21, and cell-adhesive part 22 extending continuously or intermittently (FIG. 28 shows an example of extending continuously) along periphery P of the non-cell-adhesive part 21 and surrounding the non-cell-adhesive part 21. In the cell culture substrate 1 shown in FIG. 28, a portion the surface on which cell-adhesive part 20 is arranged is referred to as “support substrate 30”.

The cell culture substrate 1 shown in FIG. 28 has support substrate 30 having one or more depressions 32, and comprises non-cell-adhesive part 21 and cell-adhesive part 22 surrounding the non-cell-adhesive part 21 on bottom surface S of each depression 32. In this embodiment, the depression 32 has round bottom surface S, though the depression 32 may have other shapes. In this embodiment, the cell-adhesive part 22 is present on the periphery of the bottom surface S of the depression 32, and the outside of the cell-adhesive part 22 is the peripheral wall surface of the depression 32, in a planar view. Therefore, cells that have adhered to the cell-adhesive part 22, when cultured, do not spread to the outside of the cell-adhesive part 22, and spread on the cell-adhesive part 22 and the non-cell-adhesive part 21 thereinside to form a cell construct.

According to the embodiment shown in FIG. 28, since cell culture parts 20 (each consisting of non-cell-adhesive part 21 and cell-adhesive part 22) are present on bottom surfaces S of depressions 32 isolated from each other in the horizontal direction, cells within each cell culture part 20 are cultured without forming intercellular bonds with cells in other cell culture parts 20 adjacent thereto, easily constructing an experimental system with high reproducibility. In this embodiment, a plurality of depressions 32, if present, are isolated from each other and arranged apart from each other with a distance of preferably 0.20 mm or more and more preferably 0.30 mm or more.

Cell culture substrate 1 according to one embodiment of the present disclosure shown in FIG. 29 has surface S comprising cell culture part 20. The cell culture part 20 has non-cell-adhesive part 21, and cell-adhesive part 22 extending continuously or intermittently (FIG. 29 shows an example of extending continuously) along periphery P of the non-cell-adhesive part 21 and surrounding the non-cell-adhesive part 21. In the cell culture substrate 1 shown in FIG. 29, a portion the surface on which cell-adhesive part 20 is arranged is referred to as “support substrate 30”.

The cell culture substrate 1 shown in FIG. 29 has one cell culture part 20 formed throughout flat surface S of support substrate 30, and cell-adhesive part 22 is arranged on the periphery of the surface S. In this embodiment, the support substrate 30 has round surface S, though the support substrate 30 may have other shapes. In this embodiment, the cell-adhesive part 22 is present on the periphery of the surface S of the support substrate 30, and the support substrate is absent outside the cell-adhesive part 22, in a planar view. Therefore, cells that have adhered to the cell-adhesive part 22, when cultured, do not spread to the outside of the cell-adhesive part 22, and spread on the cell-adhesive part 22 and the non-cell-adhesive part 21 thereinside to form a cell construct.

According to the embodiment shown in FIG. 29, since only cell culture part 20 (consisting of non-cell-adhesive part 21 and cell-adhesive part 22) is present in one cell culture substrate 1, cells within the cell culture part 20 are cultured without forming intercellular bonds with other cells, easily constructing an experimental system with high reproducibility.

### <3. Kit>

Alternative one or more embodiments of the present disclosure relate to a kit for cell culture comprising the cell culture substrate.

Features of the cell culture substrate in the kit are as already mentioned.

The kit may further comprise one or more members selected from a medium and a precoating treatment agent.

As a medium, a medium that can be used for culturing cells to be cultured and in particular, stem cells or cancer cells mentioned later, is preferable. A preferable medium for culturing stem cells can be selected from the scope of examples of a medium that can be used in a method for producing a cell construct comprising small intestinal epithelial cells.

A precoating treatment agent is a component for promoting adhesion of stem cells to cell-adhesive parts when applied in advance to a cell culture substrate. Examples of the precoating treatment agent include extracellular matrixes (collagen, fibronectin, proteoglycan, laminin, and vitronectin), gelatin, lysine, peptides, and gel matrixes containing any thereof, serum, and other substances. The precoating treatment agent may be contained in the kit in the form of a liquid composition of the precoating treatment agent dissolved or suspended in an appropriate solvent.

### <4. Stem Cell>

Stem cells used in one or more embodiments of the present disclosure can be stem cells having the ability to differentiate into small intestinal epithelial cells and are preferably stem cells having the ability to differentiate into endodermal cells (small intestinal epithelial cells, etc.), ectodermal cells and mesodermal cells, and more preferably pluripotent stem cells. Particularly, embryonic stem cells (ES cells) or induced pluripotent stem cells (iPS cells) are appropriate as the pluripotent stem cells.

Embryonic stem cells (ES cells) used in one or more embodiments of the present disclosure are mammal-derived ES cells. For example, it is possible to use ES cells derived from a rodent such as a mouse or a primate such as a human. Particularly preferably, mouse- or human-derived ES cells are used. ES cells are cells of stem cell lines generated from internal cell mass belonging to a part of an embryo in the blastocyst stage that is the initial stage of animal development. ES cells can grow almost infinitely while maintaining pluripotent differentiation capacity to theoretically differentiate into all tissues. As ES cells, for example, it is possible to use cells in which a reporter gene is introduced in the vicinity of the Pdx1 gene in order to facilitate the confirmation of the degree of differentiation. For example, the 129/Sv-derived ES cell line having cells in which the LacZ gene is incorporated at the Pdx1 locus or the ES cell line SK7 having the GFP reporter transgene under Pdx1 promoter regulation can be used. Alternatively, it is also possible to use the ES cell line PH3 having the mRFP1 reporter transgene under Hnf3βendoderm-specific enhancer fragment regulation and the GFP reporter transgene under Pdx1 promoter regulation. It is also possible to use the ES cell lines SEES1, SEES2, SEES3, SEES4, SEES5, SEES6, and SEES7, which were generated in the Department of Cell Engineering, Department of Reproductive Biology of the National Center for Child Health and Development (NCCHD) disclosed in Akutsu H, et al. Regen Ther. 2015; 1:18-29, and cell lines obtained by introducing additional genes into these ES cell lines.

Induced pluripotent stem cells (iPS cells) used in one or more embodiments of the present disclosure are pluripotent cells obtained by reprogramming somatic cells. Production of induced pluripotent stem cells have been achieved by several groups such as the group led by Professor Shinya Yamanaka at Kyoto University, the group of Rudolf Jaenisch et al. at the Massachusetts Institute of Technology, the group of James Thomson et al. at the University of Wisconsin-Madison, and the group of Konrad Hochedlinger et al. at Harvard University. For example, International publication no. WO2007/069666 discloses nuclear reprogramming factors for somatic cells containing gene products of the Oct family gene, the Klf family gene, and the Myc family gene and nuclear reprogramming factors for somatic cells containing gene products of the Oct family gene, the Klf family gene, the Sox family gene, and the Myc family gene, and further discloses a method for producing induced pluripotent stem cells by nuclear reprogramming of somatic cells, comprising a step of bringing nuclear reprogramming factors into contact with somatic cells.

Types of somatic cells used for preparing iPS cells are not particularly limited, and arbitrary somatic cells can be used. In other words, somatic cells according to an embodiment of the present disclosure include all cells constituting the living body other than germ cells, and may be differentiated somatic cells or undifferentiated stem cells. The origin of somatic cells may be, but is not particularly limited to, any of mammals, birds, fish, reptiles, and amphibians. It is preferably a mammal (for example, a rodent such as a mouse or a primate such as a human) and particularly preferably a mouse or a human. In addition, in a case in which human somatic cells are used, somatic cells of either a fetus, newborn, or adult may be used. Specific examples of somatic cells include fibroblasts (e.g., skin fibroblasts), epithelial cells (e.g., gastric epithelial cells, hepatic epithelial cells, and alveolar epithelial cells), endothelial cells (e.g., blood vessel cells and lymph vessel cells), nerve cells (e.g., neurons and glial cells), pancreatic cells, blood cells, bone marrow cells, muscle cells (e.g., skeletal muscle cells, smooth muscle cells, and cardiomyocytes), hepatic parenchymal cells, nonhepatic parenchymal cells, adipocytes, osteoblasts, cells constituting periodontal tissue (e.g., periodontal ligament cells, cementoblasts, gingival fibroblasts, osteoblasts), and cells constituting the kidneys, eyes, or ears.

The term “iPS cells” refers to stem cells having long-term self-renewal ability under predetermined culture conditions (for example, under conditions for culturing ES cells) and multilineage potential capable of differentiating into ectoderm, mesoderm, and endoderm under predetermined differentiation induction conditions. In addition, iPS cells according to an embodiment of the present disclosure may be stem cells having ability to form a teratoma when transplanted into a test animal such as a mouse.

In order to produce iPS cells from somatic cells, first, at least one type of reprogramming gene is introduced into somatic cells. The term “reprogramming gene” refers to a gene encoding a reprogramming factor functioning to reprogramming somatic cells to iPS cells. Specific examples of a combination of reprogramming genes include, but are not limited to, the following combinations.

(i) Oct gene, Klf gene, Sox gene, Myc gene
(ii) Oct gene, Sox gene, NANOG gene, LIN28 gene
(iii) Oct gene, Klf gene, Sox gene, Myc gene, hTERT gene, SV40 largeT gene
(iv) Oct gene, Klf gene, Sox gene
<5. Cell Other than Stem Cell>

Cells to be cultured using a cell culture substrate or a kit according to an embodiment of the present disclosure are not limited to stem cells and may be other cells. For example, other cells may be cancer cells.

The origin organism species of cancer cells is not particularly limited. Examples of human-derived cells include large intestinal epithelial cancer-derived Caco-2 cells, liver cancer-derived HepG2 cells and HepaRG cells, breast cancer-derived MCF-7 cells, lung cancer-derived A-549 cells, uterine cervical cancer-derived HeLa cells, and skin cancer-derived A-431 cells. Particularly, cancer cells that easily form cyst-like sac-shaped tissues in vivo, for example, various pancreatic cancer cells, ovary cancer cells, or kidney cancer cells, can be used.

### <6. Sac-Shaped Cell Construct>

A sac-shaped cell construct can be induced by culturing cells using a cell culture substrate or a kit according to an embodiment of the present disclosure.

One or more embodiments of the present disclosure are based on the surprising finding that when cells are cultured on a cell culture substrate according to an embodiment of the present disclosure, cells accumulate with a high density in a cell-adhesive part extending so as to surround a non-cell-adhesive part, to obtain a cell construct having a sac-shaped structure where the resulting cells are distributed on the outer circumference.

Particularly, in the case of culturing stem cells on a cell culture substrate according to an embodiment of the present disclosure, a sac-shaped cell construct comprising small intestinal epithelial cells can be induced in the outer circumference. The sac-shaped cell construct comprising small intestinal epithelial cells obtained by the differentiation induction of stem cells can be used as a gut organoid.

Also, in the case of culturing cancer cells on a cell culture substrate according to an embodiment of the present disclosure, a sac-shaped cell construct containing a liquid component can be induced. The sac-shaped cell construct induced by culturing cancer cells on a cell culture substrate according to an embodiment of the present disclosure has a cancer cyst-like structure and is therefore useful in the development of drugs for preventing or treating cancers or pathological studies on cancers. For culturing cancer cells to prepare cyst-like tissues, it has heretofore been required to perform gel-embedded three-dimensional culture. Nonetheless, a sac-shaped cell construct can be induced from cancer cells by a convenient method of culturing cancer cells on a cell culture substrate according to an embodiment of the present disclosure.

The whole shape of the sac-shaped cell construct produced by the culture and differentiation induction of cells using a cell culture substrate or a kit according to an embodiment of the present disclosure is not particularly limited but it is usually granular. The word “granular” also encompasses “spherical”.

### <7. Method for Producing Sac-Shaped Cell Construct>

A sac-shaped cell construct can be produced using a cell culture substrate according to an embodiment of the present disclosure. This production method comprises, for example:

seeding cells onto a cell culture substrate having the features described above; and

culturing the cells seeded to induce a sac-shaped cell construct.

Examples of the cells can include stem cells and cancer cells.

For use in the production of a cell construct, a cell culture substrate according to an embodiment of the present disclosure is preferably subjected to precoating treatment with a precoating treatment agent in order to promote adhesion of cells to cell-adhesive parts. Specific examples of the precoating treatment agent are as already mentioned. By performing precoating treatment, it is possible to promote adhesion of cells having low adhesiveness to cell-adhesive parts and effectively carry out adhesion culture of cells.

The step of culturing the cells seeded in the cell culture substrate so as to differentiate into a sac-shaped cell construct can be performed in a medium that permits growth of cells and induction of a sac-shaped cell construct therefrom. The medium may be a serum-containing medium, or may be a serum-free medium containing a known component having a property of replacing serum. As a medium, MEM medium, BME medium, DMEM medium, DMEM-F12 medium, αMEM medium, IMDM medium, ES medium, DM-160 medium, Fisher medium, F12 medium, WE medium and RPMI1640 medium, etc. can be used. It is possible to add various growth factors, antibiotics, amino acids, and other additives to a medium. A preferable medium for culturing stem cells will be mentioned later.

The seeding density of cells on a cell culture substrate is not particularly limited as long as it complies with an ordinary method. For example, cells are seeded on a cell culture substrate at a density of preferably 3×10⁴ cells/cm² or more, more preferably 3×10⁴ to 5×10⁵ cells/cm², and further preferably 3×10⁴ to 2.5×10⁵ cells/cm².

The culture temperature is usually 37° C. It is preferable to carry out the culture under a CO₂ concentration atmosphere of about 5% by using an appropriate cell culture apparatus such as a CO₂ cell culture apparatus.

The culture period of cells after seeding to the cell culture substrate differs depending on the initial seeding density of cells and the shape and size of a cell-adhesive part but is preferably on the order of 2 to 4 weeks.

Although a sac-shaped cell construct induced from cells on a cell culture substrate according to an embodiment of the present disclosure is detached by spontaneous floating from a cell culture substrate, the detachment of the cell construct from the cell culture substrate may be promoted by use of various approaches, such as mild enzymatic treatment (e.g., Accutase and TrypLE), EDTA treatment, flowing of a liquid such as a medium, and physical detachment with a scraper, which do not disrupt the cell construct.

After detachment of the sac-shaped cell construct from the cell culture substrate, suspension culture may be further continued. The period of the suspension culture is not limited.

### <8. Cell Construct Comprising Small Intestinal Epithelial Cell Obtained by Differentiation Induction of Stem Cells>

The intestine is a complex organ including cells derived from all 3 germ layers (endoderm, ectoderm, and mesoderm). The intestine, which is composed of endoderm-derived small intestinal epithelial cells (e.g., intestine cells, goblet cells, endocrine cells, brush cells, Paneth cells, and M cells), mesoderm-derived lymph tissue, smooth muscle cells, interstitial cells of Cajal, ectoderm-derived intestinal nerve plexus, and other types of cells in a complex manner, has a variety of functions including secretion, absorption, and peristaltic movement.

A tissue obtained by the method described in Patent Literature 1 contains only small intestinal epithelial cells. Since activin is used in the induction of differentiation of embryonic stem cells, only cells derived from a substantially single germ layer, i.e., endoderm-derived cells therein, are formed. Hence, for obtaining intestine tissues containing other types of cells derived from mesoderm or ectoderm, it is necessary to separately induce differentiation into these other types of cells, as disclosed in Non Patent Literature 1. The method described in Patent Literature 1 comprises the step of culturing cells embedded in Matrigel for the induction of epithelial differentiation and thus has problems associated with productivity in this respect.

The method described in Non Patent Literature 2 disadvantageously requires labor for culture.

Since a tissue obtained by the method described in Patent Literature 2 contains only small intestinal epithelial cells, this method also has problems similar to those of the method described in Patent Literature 1.

Meanwhile, according to the method described in Patent Literature 3 and Non Patent Literature 3, differentiation not only into intestinal epithelial tissues but into muscular tissues, nervous tissues, and the like can be induced by single culture. This method has high production efficiency because many intestinal tissues can be cultured at the same time on one substrate on which many patterns are formed. The method is also easily applicable to transplantation because the culture is achieved without the use of organism-derived materials.

However, a problem of the method described in Patent Literature 3 and Non Patent Literature 3 is drastic reduction in the yields of tissues of interest depending on the type of cells to be cultured, and requires improving the yields of tissues of interest by improvement in culture method.

Specifically, there has been a demand for an approach for efficiently producing a cell construct comprising small intestinal epithelial cells, irrespective of the origin of cultured cells.

Accordingly, as a solution to the problem described above, one or more embodiments of the present disclosure include the production of a cell construct comprising small intestinal epithelial cells by the culture and differentiation induction of stem cells using a cell culture substrate or a kit according to an embodiment of the present disclosure.

The small intestinal epithelial cells are typically small intestinal epithelial cells expressing a trophectodermal cell marker.

One or more embodiments of the present disclosure are based on the surprising finding that cells accumulate with a high density in a cell-adhesive part extending so as to surround a non-cell-adhesive part (central part) during stem cell culture, and differentiate into small intestinal epithelial cells expressing a trophectodermal cell marker, to obtain a cell construct having a sac-shaped structure where the resulting cells are distributed on the outer circumference. In Examples, it has been confirmed that cytokeratin 7, a marker of trophectodermal cells, and CDX2, a marker of small intestinal epithelial cells and trophectodermal cells, were strongly expressed in cells that accumulated in the cell-adhesive part.

The small intestinal epithelial cells can be confirmed based on the expression of transcription factors CDX2 and HNF4 in cell nuclei and villin in villi and the expression of an endodermal marker E-cadherin, etc. The presence of these markers can be detected by immunohistochemical staining using antibodies, PCR evaluation using mRNA, or the like.

A cell construct according to one or more embodiments produced by the culture and differentiation induction of stem cells using a cell culture substrate or a kit according to an embodiment of the present disclosure comprises small intestinal epithelial cells and is useful as a gut organoid having gut-like functions. The term “gut organoid” refers to a cell construct (tissue) having functions (specifically, for example, a motile movement function, a mucus secretion function, and a substance absorption function) similar to those of an intestine of the origin organism of cells, and in particular, an intestine of a mammal such as a human or a human intestine. The cell construct produced by the culture and differentiation induction of stem cells using a cell culture substrate or a kit according to an embodiment of the present disclosure is useful in the development of drugs for preventing or treating intestinal diseases or pathological studies on intestinal diseases.

The whole shape of the cell construct produced by the culture and differentiation induction of stem cells using a cell culture substrate or a kit according to an embodiment of the present disclosure is not particularly limited but it is usually granular. The word “granular” also encompasses “spherical”.

As in Non Patent Literature 4, strong expression of CDX2 indicates that mouse ES cells differentiate into trophectodermal cells. In Examples of the present disclosure, it has been confirmed that a marker CDX2 or cytokeratin 7 was strongly expressed in an accumulating portion, also suggesting differentiation into cells having the properties of trophectodermal cells.

Non Patent Literature 5 states that a tissue having a sac-shaped structure containing CDX2-positive cells is obtained from human iPS cells. Accordingly, in one or more embodiments of the present disclosure, the possibility is suggested that in an accumulating portion having dense accumulations of cells on a cell-adhesive part, stem cells differentiate by the culture of the stem cells into CDX2-positive cells, which also corresponds to the formation of a sac-shaped structure.

Besides, Non Patent Literatures 6 to 8 also disclose that ES cells or iPS cells have the ability to differentiate into trophectodermal cells.

As shown in these past findings and later in the present disclosure, decrease in the expression of Oct3/4 gene has been observed in cells that have accumulated in a cell-adhesive part. Therefore, in culture using a cell culture substrate according to an embodiment of the present disclosure, it is predicted that stem cells attach and grow within a cell-adhesive part to form an accumulating portion where the stem cells differentiate into small intestinal epithelial cells expressing a trophectodermal cell marker.

In studies shown in Non Patent Literature 9, decreased expression of Oct gene indicates that mouse ES cells differentiate into trophectoderm. It is predicted that similar differentiation also occurs in a cell construct produced using a cell culture substrate or a kit according to an embodiment of the present disclosure.

A cell construct produced by the culture and differentiation induction of stem cells using a cell culture substrate or a kit according to an embodiment of the present disclosure more preferably comprises endodermal cells, ectodermal cells and mesodermal cells.

The endoderm forms the digestive tract and tissues of organs, such as the lung, thyroid, pancreas and liver, and cells of secretory glands opening into the digestive tract, peritoneum, pleura, larynx, eustachian tube, trachea, bronchial tube, urinary tract (the bladder, most of the urethra, part of the ureter), and other tissues. Differentiation from ES cells or iPS cells into endodermal cells can be confirmed by measuring expression levels of endoderm-specific genes. Examples of endoderm-specific genes include AFP, SERPINAL SST, ISL1, IPF1, IAPP, EOMES, HGF, ALBUMIN, PAX4, and TAT.

Particular endodermal cells that may be contained in a cell construct produced by the culture and differentiation induction of stem cells using a cell culture substrate or a kit according to an embodiment of the present disclosure include small intestinal epithelial cells. The gut organoid preferably contains, as small intestinal epithelial cells, at least one type of cells selected from enterocytes, goblet cells, enteroendocrine cells, and Paneth cells, and particularly preferably contain, as small intestinal epithelial cells, all of enterocytes, goblet cells, enteroendocrine cells, and Paneth cells. It is possible to determine the presence of endodermal cells in a cell construct produced using a cell culture substrate or a kit according to an embodiment of the present disclosure based on the positive expression of endodermal cell markers. The enterocyte marker may be CDX2, the goblet cell marker may be MUC2, the enteroendocrine cell marker may be CGA, and the Paneth cell marker may be DEFA6. In addition to the above, ECAD, Na⁺/K⁺-ATPase, and villin are intestinal epithelial cell markers. Further, it is also possible to use the definitive endoderm markers FOXA2, SOX17, and CXCR4 as markers for detecting endodermal cells. The primary endoderm and mesoderm markers GATA4, GATA6, and T (Brachyury) also can be used as markers for detecting endodermal cells.

The ectoderm forms several tissues including, for example, skin epidermis, epithelium of the urethral end of a man, hair, nails, skin glands (including mammary gland and sweat gland), sensory organs (oral cavity, pharynx, nose, epithelium at the distal end of the rectum, salivary gland) and lens. A part of the ectoderm is invaginated in a groove shape in the developmental process to form a neural tube, which is also a source of neurons and melanocytes of the central nervous system such as the brain and spinal cord. The ectoderm also forms the peripheral nervous system. Differentiation from ES cells or iPS cells into ectodermal cells can be confirmed by measuring expression levels of ectoderm-specific genes. Examples of ectoderm-specific genes may include β-TUBLIN, NESTIN, GALANIN, GCM1, GFAP, NEUROD1, OLIG2, SYNAPTPHYSIN, DESMIN, and TH.

Particular ectodermal cells that may be contained in a cell construct produced by the culture and differentiation induction of stem cells using a cell culture substrate or a kit according to an embodiment of the present disclosure include cells that constitute the intestinal nerve plexus. It is possible to determine the presence of ectodermal cells in a cell construct produced using a cell culture substrate or a kit according to an embodiment of the present disclosure based on the positive expression of ectodermal cell markers. As markers for detecting ectodermal cells, the intestinal nerve plexus marker PGP9.5 and the neural progenitor marker SOX1 can be used.

The mesoderm forms a body cavity and mesothelium lining inside thereof, muscles, skeletons, skin dermis, connective tissue, heart, blood vessels (including vascular endothelium), blood (including blood cells), lymph vessels, spleen, kidneys, ureters, and gonad (testis, uterus, and gonadal epithelium). Differentiation from ES cells or iPS cells into mesodermal cells can be confirmed by measuring expression levels of mesoderm-specific genes. Examples of mesoderm-specific genes may include FLK-1, COL2A1, FLT1, HBZ, MYF5, MYOD1, RUNX2, and PECAM1.

Particular mesodermal cells that may be contained in a cell construct produced by the culture and differentiation induction of stem cells using a cell culture substrate or a kit according to an embodiment of the present disclosure include smooth muscle cells and interstitial cells of Cajal. It is possible to determine the presence of mesodermal cells in gut organoids based on the positive expression of mesodermal cell markers. As mesodermal cell markers, the smooth muscle cell marker α-smooth muscle actin (SMA) and the Cajal cell markers CD34 and CKIT (for double-positive cells) can be used. The primary endoderm and mesoderm markers GATA4, GATA6, and T (Brachyury) also can be used as markers for detecting mesodermal cells.

A cell construct produced by the culture and differentiation induction of stem cells using a cell culture substrate or a kit according to an embodiment of the present disclosure preferably comprises small intestinal epithelial cells in at least a part of the outer surface thereof. According to this embodiment, substances outside of the cell construct can be absorbed into the inside via small intestinal epithelial cells on the outer surface thereof, which is preferable.

### <9. Method for Producing Cell Construct Comprising Small Intestinal Epithelial Cell>

A cell construct comprising small intestinal epithelial cells can be produced using a cell culture substrate according to an embodiment of the present disclosure. This production method comprises, for example:

seeding stem cells onto a cell culture substrate having the features described above; and

culturing the stem cells seeded to differentiate a part of the stem cells into small intestinal epithelial cells.

For use in the production of a cell construct, a cell culture substrate according to an embodiment of the present disclosure is preferably subjected to precoating treatment with a precoating treatment agent in order to promote adhesion of stem cells to cell-adhesive parts. Specific examples of the precoating treatment agent are as already mentioned. By performing precoating treatment, it is possible to promote adhesion of stem cells having low adhesiveness to cell-adhesive parts and effectively carry out adhesion culture and differentiation induction of cells.

Stem cells can be cultured under conditions that allow them to remain undifferentiated before seeding. A medium for use in this culture is not particularly limited as long as it is a medium that does not allow differentiation induction of stem cells. Examples thereof include a medium containing a leukemia inhibitory factor which are known to have a feature of allowing mouse embryonic stem cells and mouse induced pluripotent stem cells to remain undifferentiated and a medium containing basic FGF which are known to have a feature of allowing human iPS cells to remain undifferentiated.

The step of culturing the stem cells seeded in the cell culture substrate to differentiate a part of them into small intestinal epithelial cells can be performed in a medium that permits growth and differentiation induction of stem cells. The medium is not particularly limited. Specific examples thereof include media used in Patent Literature 3 and Non Patent Literature 3, and commercially available media such as StemFit (Ajinomoto Co., Inc.), StemFlex (Life Technologies Corp.), and ReproFF (ReproCELL Inc.). The medium may be a serum-containing medium, or may be a serum-free medium containing a known component having a property of replacing serum.

As a medium, MEM medium, BME medium, DMEM medium, DMEM-F12 medium, αMEM medium, IMDM medium, ES medium, DM-160 medium, Fisher medium, F12 medium, WE medium and RPMI1640 medium, etc. can be used. It is possible to add various growth factors, antibiotics, amino acids, and other additives to a medium. For example, 0.1 to 2% pyruvic acid, 0.1 to 2% nonessential amino acids, 0.1 to 2% penicillin/streptomycin, 0.1 to 1% glutamine, 0.1 to 2% β mercaptoethanol, and a 1 mM to 20 mM ROCK inhibitor (e.g., Y-27632) may be added.

The seeding density of stem cells on a cell culture substrate is not particularly limited as long as it complies with an ordinary method. For example, stem cells are seeded on a cell culture substrate at a density of preferably 3×10⁴ cells/cm² or more, more preferably 3×10⁴ to 5×10⁵ cells/cm², and further preferably 3×10⁴ to 2.5×10⁵ cells/cm².

The culture temperature is usually 37° C. It is preferable to carry out the culture under a CO₂ concentration atmosphere of about 5% by using an appropriate cell culture apparatus such as a CO₂ cell culture apparatus.

The culture period of stem cells after seeding to the cell culture substrate differs depending on the initial seeding density of cells and the shape and size of a cell-adhesive part but is preferably on the order of 2 to 4 weeks. The present inventors have found that: when stem cells are cultured on a cell culture substrate having a structure described herein and induced to differentiate, a cell construct comprising small intestinal epithelial cells induced to differentiate are detached by spontaneous floating in 2 to 4 weeks after seeding and can be collected; and the collection rate of the cell construct thus collected is markedly high. On a substrate having a round cell-adhesive part described in Non Patent Literature 3, a cell construct is detached after a lapse of 30 days or more, and furthermore, its collection rate is very low. In contrast to this, a method according to an embodiment of the present disclosure is advantageous.

Although a cell construct obtained by the differentiation induction of stem cells on a cell culture substrate according to an embodiment of the present disclosure is detached by spontaneous floating from a cell culture substrate, the detachment of the cell construct from the cell culture substrate may be promoted by use of various approaches, such as mild enzymatic treatment (e.g., Accutase and TrypLE), EDTA treatment, flowing of a liquid such as a medium, and physical detachment with a scraper, which do not disrupt the cell construct.

After detachment of the cell construct from the cell culture substrate, suspension culture may be further continued. The period of the suspension culture is not limited.

Hereinafter, the present disclosure will be described with reference to specific experimental results. However, the scope of the present disclosure is not limited by the experimental results.

### EXAMPLES

### Example 1

### (Production of Cell Culture Substrate)

A cell culture substrate having a cell-adhesive part made of an annular pattern having an inside diameter of 180 μm, 280 μm or 380 μm and a width of 60 μm (see FIG. 1), which was a region formed by the oxidation decomposition of a layer of polyethylene glycol 400 formed on a glass substrate, and a non-cell-adhesive part which was a region of the surface (inside and outside the annular pattern of the cell-adhesive part) of the glass substrate coated with polyethylene glycol (PEG400) was produced. The cell culture substrate has a plurality of cell-adhesive parts formed at intervals of 300 to 500 μm and made of the annular pattern (see FIG. 1). In the description below, the cell-adhesive part made of the annular pattern is referred to as “annular cell-adhesive part”.

The cell culture substrate was produced by the procedures described in the patent literature and the non patent literature described above. The summary thereof will be described below.

### (First Stage Reaction)

39.0 g toluene, 0.48 g epoxysilane TSL8350 (manufactured by GE Toshiba Silicones Co., Ltd.), and 0.97 g triethylamine were mixed and stirred at room temperature for 10 minutes. A 10-cm square glass substrate, which was previously UV-cleaned, was immersed in the silane solution such that its surface to be washed turned up. After being left at room temperature for 16 hours, the substrate was washed with ethanol and water and dried in nitrogen blow. A thin film containing an epoxy group was thereby formed on the glass substrate surface.

### (Second Stage Reaction)

Polyethylene glycol having an average molecular weight of 400 (PEG400) in an amount of 50 g was stirred, during which concentrated sulfuric acid was added dropwise in an amount of 25 μl. The resulting mixture was stirred as it was for several minutes, and the whole amount was then transferred to a glass dish. The above-described substrate was immersed therein so as to allow reaction to proceed at 80° C. for 20 minutes. After the reaction, the substrate was washed well with water and dried in nitrogen blow. A uniform hydrophilic thin film was thereby formed on the glass surface.

### (Oxidation Treatment)

A photomask having a titanium oxide photocatalyst applied on the whole surface was produced. The photomask used was in a 5-inch size and had openings with shapes corresponding to the plurality of annular cell-adhesive parts with the above-described size at intervals of 300 to 500 μm. The photomask also had openings having a width of about 1.5 cm located therearound. The illuminance of a light exposure machine was measured in advance at a wavelength of 350 nm and used as a guideline for the setting of light exposure time. The photocatalyst layer of this photomask was contacted with the hydrophilic thin film of the substrate surface, and the resultant was loaded in the light exposure machine so as to be irradiated with light from the photomask side. The hydrophilic thin film of the substrate surface was partially oxidation-decomposed by light exposure for 50 seconds with a mercury lamp having an illuminance of 20 mW/cm² at a wavelength of 350 nm. This substrate was cut into a size of 25 mm×15 mm and used as a cell culture substrate. The cell culture substrate was subjected to EOG sterilization treatment for 22 hours before being used in cell culture.

The cell culture substrate was disposed on the bottom surface of a 3.5 cm petri dish (Corning Inc.), coated by contact with vitronectin (Life Technologies Corp.) diluted 1/100 with phosphate-buffered saline (PBS) at room temperature for 30 minutes or longer, then washed with PBS three times, and then used.

The cell culture substrate thus obtained has a cross-sectional structure as shown in FIG. 1(B).

### (Culture)

The Department of Cell Engineering, Department of Reproductive Biology of NCCHD established Edom iPS cells as a human iPS cell line by causing cells obtained from menstrual blood to transiently express four Yamanaka factors with Sendai virus vectors (PLoS Genet. 2011 May; 7 (5): e1002085. Published online 2011 May 26. doi: 10.1371/journal.pgen.1002085PMCID: PMC3102737). Edom iPS cells were allowed to grow in advance using StemFit medium (Ajinomoto Co., Inc.) in a vitronectin-coated dish for cell culture (Corning Inc.). The cells thus allowed to grow were detached from the dish by treatment with EDTA (Invitrogen Corp.) diluted 1/1000 with PBS at 37° C. for 10 minutes, then seeded at 1×10⁶ cells to the cell culture substrate, and cultured. As a medium, the XF hESC medium described in Non Patent Literature 3 was used. On the day of seeding, the medium contained Y27632 but was replaced with the medium free from Y27632 on the following day and maintained. On day 4 or later, medium replacement was performed once every 3 to 4 days. During culture, tissues spontaneously detached from the cell culture substrate were collected, and maintained by suspension culture using the same medium as above in another petri dish.

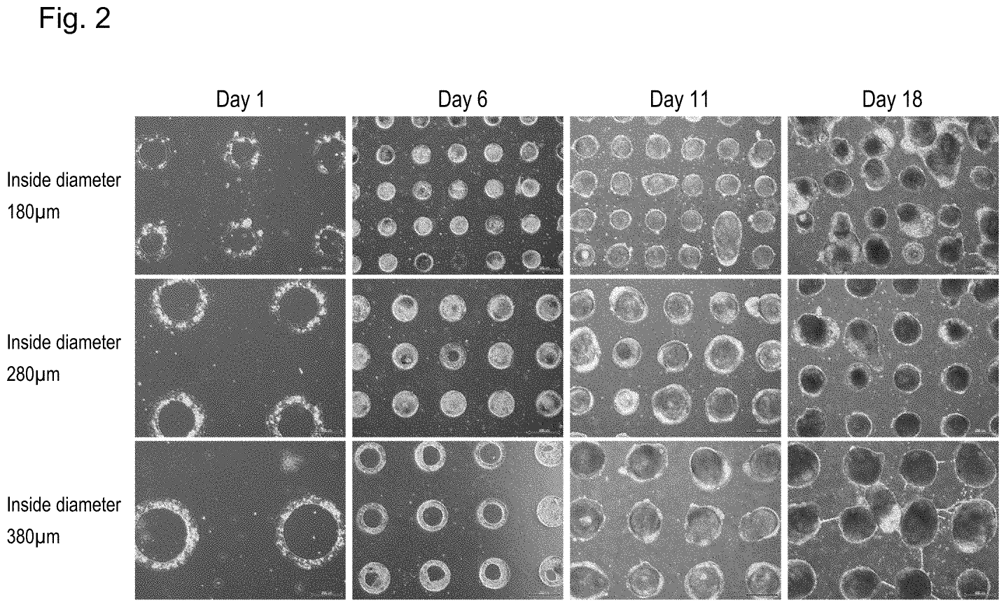

FIG. 2 shows observation images of cultures on culture days 1, 6, 11 and 18 obtained by culture using each cell culture substrate having an annular cell-adhesive part having a different inside diameter. The photographs of culture day 1 have a higher magnification factor than that of the others. As observed, cells grew first in the annular cell-adhesive part, and then, the inside non-cell-adhesive part surrounded by the annular cell-adhesive part was covered with the cells that grew.

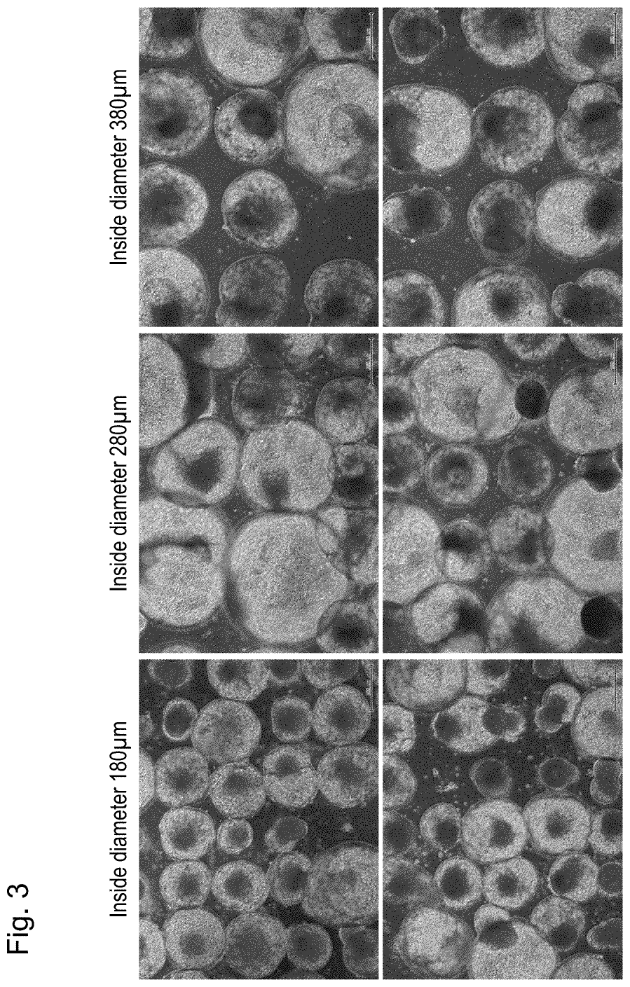

FIG. 3 shows observation images of cultures on culture week 3. The observation images shown in FIG. 3 show that tissues having a sac-shaped structure were formed at a high proportion by culture on each cell culture substrate.

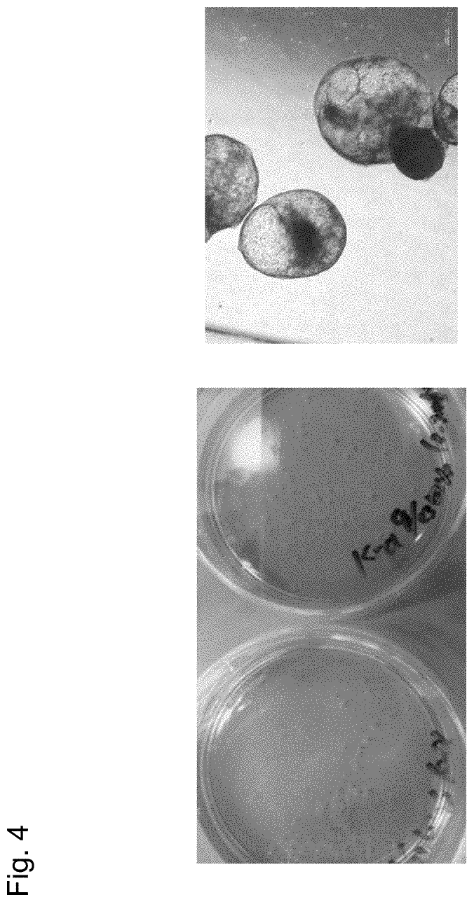

The results of this culture showed that tissues having a sac-shaped structure are spontaneously detached from the surface in 2 to 3 weeks after the start of culture and can be collected. FIG. 4 shows observation images of a tissue having a sac-shaped structure formed on and detached from a substrate having an annular cell-adhesive part having an inside diameter of 380 μm (the left photograph shows the whole dish, and the right photograph is an observation image of the tissue). In the left photograph of FIG. 4, white-dot like substances seen in the dish are tissues having a sac-shaped structure. The right photograph of FIG. 4 is a microscopic observation image of tissues having a sac-shaped structure obtained in this Example. The observation image of tissues having a sac-shaped structure obtained in this Example is similar to an observation image of sac-shaped tissues (gut organoids) having intestinal functions obtained by culturing iPS cells in a medium similar to that used herein, as described in Uchida et al., JCI Insight, Vol. 2, e86492 2017. From this and results of Example 4 and Example 5 mentioned later, it is evident that the tissues having a sac-shaped structure obtained in this Example are also gut organoids.

The proportion of the collected tissues having a sac-shaped structure to the number of annular cell-adhesive parts on the cell culture substrate (hereinafter, also referred to as “tissue collection rate”) was 80% or more and was thus high yields.

### Comparative Examples 1 to 3

In Comparative Example 1, the cell culture substrate described in Uchida et al., JCI Insight, Vol. 2, e86492 2017 was prepared by forming a non-cell-adhesive part which was a region coated with polyethylene glycol layer, and a plurality of round cell-adhesive parts of 1500 μm in diameter formed by the oxidation decomposition of the polyethylene glycol layer.

In Comparative Example 2, a cell culture substrate having the same structure as in Comparative Example 1 was prepared except that the respective diameters of the plurality of round cell-adhesive parts were 282 μm.

The methods for producing the cell culture substrates of Comparative Example 1 and Comparative Example 2 were similar to the method for producing the cell culture substrate of Example 1, and the shape of openings in a photomask can be appropriately changed according to the cell-adhesive parts.

In Comparative Example 3, a glass substrate provided with no adhesive pattern was prepared.

The seeding and culturing of Edom iPS cells were performed on the substrate of each of Comparative Examples 1 to 3 under the same conditions as in Example 1.

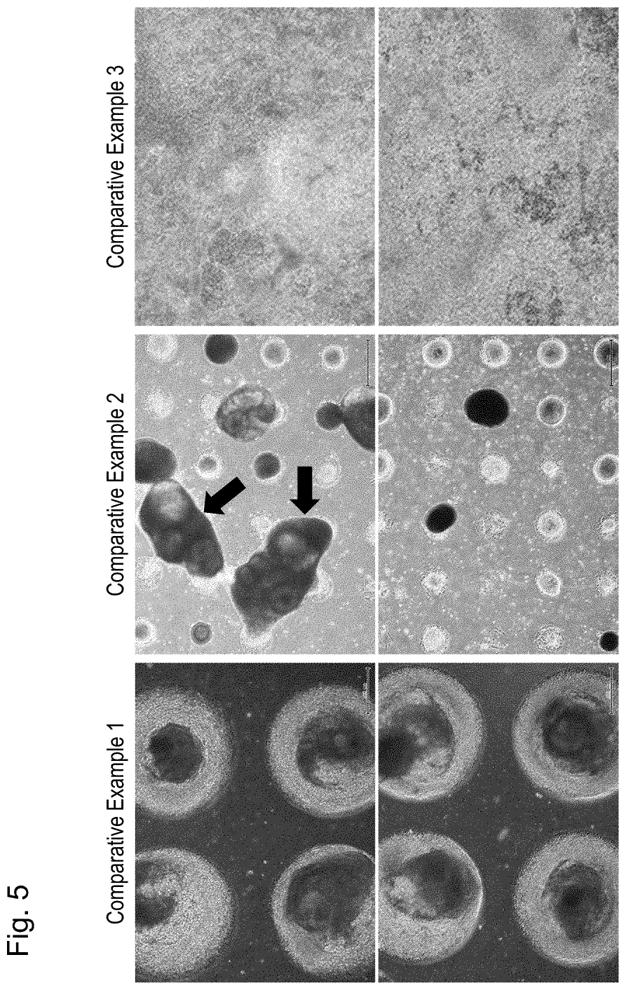

FIG. 5 shows microscopic observation images of cultures on 3 weeks after the start of culture on the substrate in each of Comparative Example 1, Comparative Example 2, and Comparative Example 3.

In Comparative Example 1, the start of detachment of tissues having a sac-shaped structure from the surface was observed 3 to 4 weeks after the start of culture, and the tissue collection rate was 4 to 5%. Comparative Example 1 compared with Example 1 described above had a long culture period until detachment and a low tissue collection rate.

In Comparative Example 2, tissues having a sac-shaped structure were obtained 2 to 3 weeks after the start of culture, whereas many cell aggregates which were dark in the observation images were obtained. The tissue collection rate of the tissues having a sac-shaped structure was 10% or less. As in the tissues indicated by arrows in “Comparative Example 2” of FIG. 5, tissues having a large-sized sac-shaped structure formed by the fusion of cultures were also formed in a plurality of adjacent round patterns.

In Comparative Example 3, there was a case in which about one or two small sac-shaped structures were obtained, whereas cell growth on the whole surface of the substrate was required before tissues having a sac-shaped structure were formed. Besides, a time of one month or more was required for culture, and the number of formed tissues having a sac-shaped structure was much smaller than that in the case of Example 1. Since the number of patterns in the cell-adhesive parts is not defined, the tissue collection rate cannot be calculated in this case.

These results show that the culture of iPS cells in a substrate having a plurality of cell-adhesive parts made of an annular pattern, as in Example 1, can yield tissues having a sac-shaped structure in a short culture period, and offers a markedly high tissue collection rate.

### Example 2

In order to perform the follow-up of the pattern culture in Example 1, the following time lapse observation was carried out.

Equipment was produced in which the cell culture substrate having a plurality of annular cell-adhesive parts having an inside diameter of 380 μm and a width of 60 μm, used in Example 1, was mounted on the bottom surface of a dish. By using this equipment, the seeding and culturing of Edom iPS cells were performed under the same conditions as in Example 1.

Photographs were taken using BioStation (Nikon Corp.) every 12 hours from culture days 4 to 21. The photographing operation followed the attached manual, and medium replacement was performed once every 2 to 3 days.

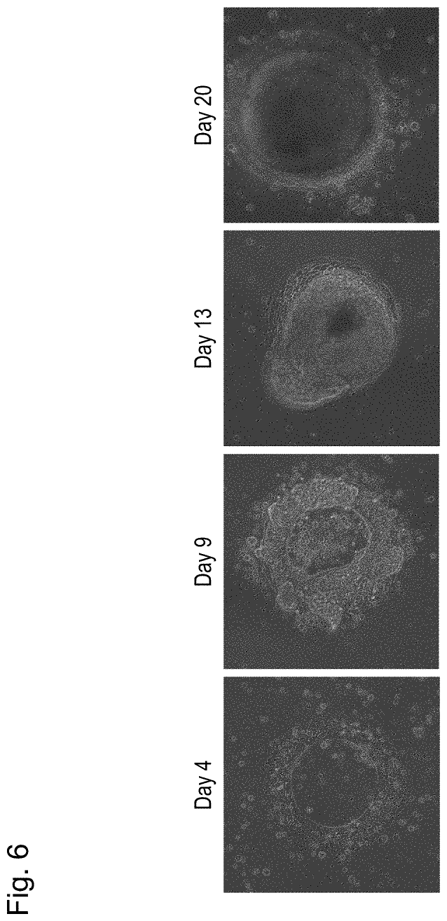

FIG. 6 shows observation images of cells around one annular cell-adhesive part on each of culture days 4, 9, 13 and 20. From this, it was confirmed that cells first grew formed a layer on the annular pattern and then formed a sac-shaped structure up to culture day 21.

### Example 3

The formation of tissues having a sac-shaped structure was attempted using other cell types.

A SEES2 cell line of human ES cells was cultured using the same cell culture substrate having annular cell-adhesive parts having an inside diameter of 180 μm, 280 μm or 380 μm and a width of 60 μm as in Example 1. First, growing culture was performed in a vitronectin-coated dish for cell culture (Corning Inc.) using StemFit medium (Ajinomoto Co., Inc.) supplemented with rhLIF (Wako Pure Chemical Industries, Ltd.) diluted 1/1000. The cells growing-cultured were collected by detachment treatment with Accutase (Life Technologies Corp.) at 37° C. for 5 minutes, then seeded to the substrate, and cultured by the same procedures as in Example 1.

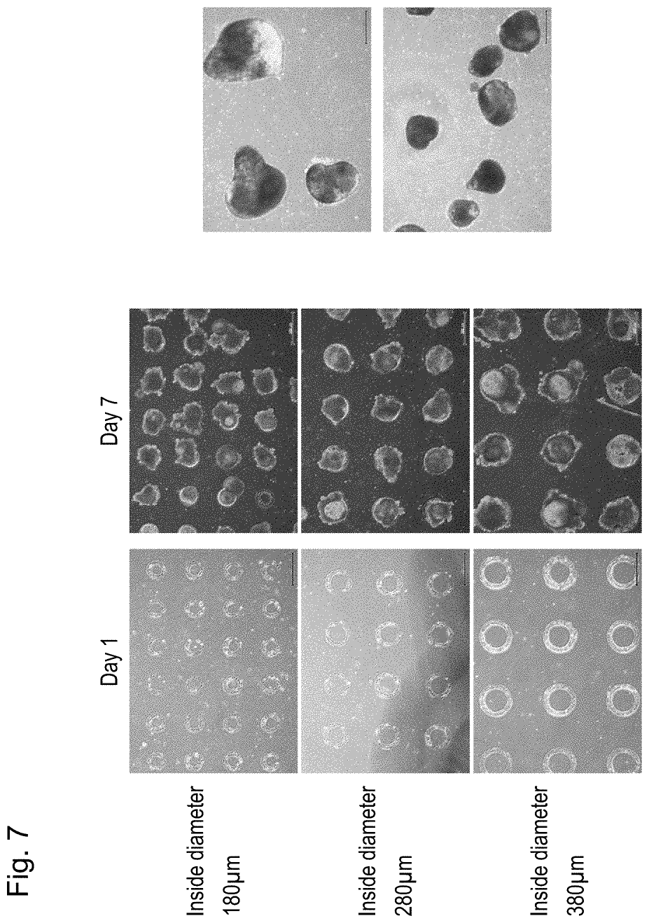

FIG. 7 shows observation images on days 1 and 7 of culture using each cell culture substrate, and observation images of tissues having a sac-shaped structure collected after 3-week culture using the cell culture substrate having annular cell-adhesive parts having an inside diameter of 280 μm. The observation images and tissue collection rate of the tissues having a sac-shaped structure were similar to those of Example 1 using iPS cells.

These results show that: the culture of stem cells on a cell culture substrate comprising an annular cell-adhesive part can efficiently produce tissues having a sac-shaped structure; and the formation of such tissues also takes place in the case of using ES cells in the same manner as in the case of using iPS cells in Example 1, irrespective of the type of pluripotent stem cells.

### Example 4

In order to study the process of culture in Example 1, cell culture was performed on the same cell culture substrate having annular cell-adhesive parts having an inside diameter of 380 μm and a width of 60 μm as in Example 1, and marker expression was examined by immunostaining.

The cell culture substrate having annular cell-adhesive parts having an inside diameter of 380 μm and a width of 60 μM, and cell culture conditions are as described in Example 1.

The cell culture substrate containing tissues on culture day 4, 7 or 12 to 14 was fixed in 4% paraformaldehyde (Wako Pure Chemical Industries, Ltd.) at room temperature for 20 minutes, then washed with PBS, and subjected to blocking operation with PBS containing 1% BSA and 0.1% Triton at room temperature for 30 minutes. Thereafter, the substrate was incubated at room temperature for 1 hour with a mouse IgG1-labeled anti-cytokeratin 7 antibody (Abcam plc, dilution ratio: 1/500), a mouse IgG3b-labeled anti-Oct3/4 antibody (Santa Cruz Biotechnologies, Inc., dilution ratio: 1/200), a rabbit IgG-labeled anti-Ki67 antibody (Abcam plc, dilution ratio: 1/500) or a rabbit IgG-labeled anti-CDX2 antibody (Abcam plc, dilution ratio: 1/1000). The substrate thus incubated was washed with PBS three times and then incubated at room temperature for 30 minutes with an Alexa 488-labeled anti-rabbit IgG antibody (Molecular Probes, dilution ratio: 1/1000) or an Alexa 546-labeled anti-mouse IgG antibody (Molecular Probes, dilution ratio: 1/1000) diluted with PBS. The substrate thus incubated was further washed with PBS three times. Thereafter, the nuclei of cells on the substrate were stained with DAPI (Sigma-Aldrich Co. LLC, dilution ratio: 1/1000) at room temperature for 10 minutes, then mounted, and observed under a confocal microscope. The types of the antibodies were appropriately chosen.

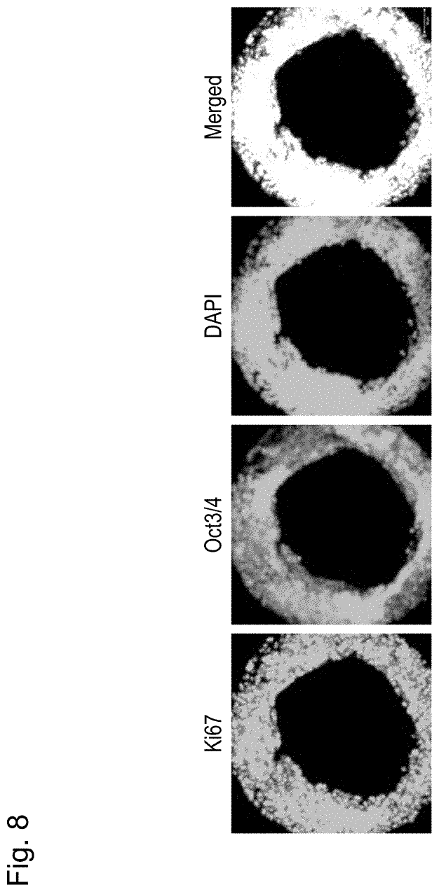

FIG. 8 shows observation images on culture day 4. As a result, Ki67-positive and Oct3/4-positive undifferentiated cells having the ability to grow were present on the annular cell-adhesive part on culture day 4, suggesting that an accumulating portion can be formed.

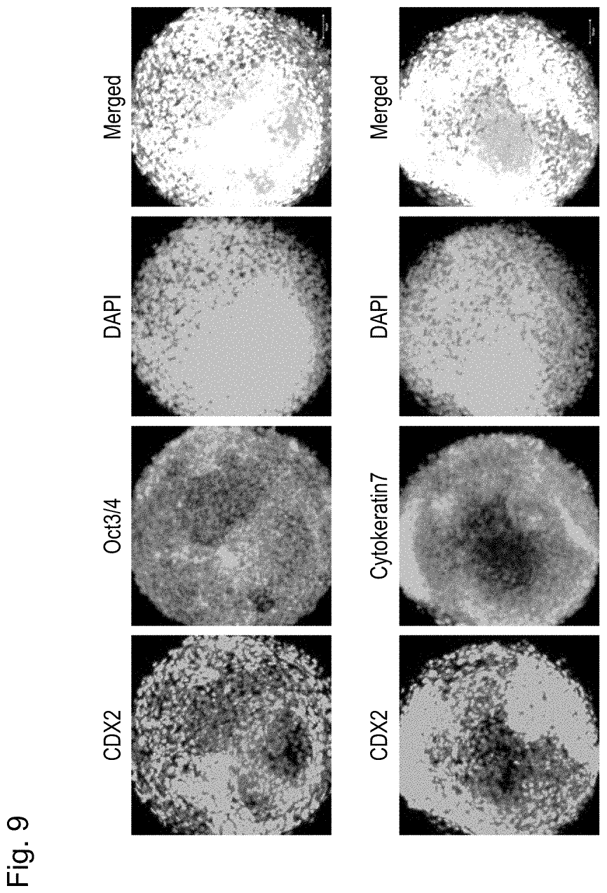

FIG. 9 shows observation images on culture day 7. The upper observation images of FIG. 9 show that tissues were formed in which Oct3/4-positive undifferentiated cells having multilineage potential were preset mainly in the inside and CDX2-positive cells were present in the outer circumference. The lower observation images of FIG. 9 show that the CDX2-positive cells were cytokeratin 7-positive trophectodermal cells.

These results suggested that in tissues formed by culturing pluripotent stem cells on the cell culture substrate having annular cell-adhesive parts of Example 1, a particularly dense portion (an accumulating portion) formed by cells in the outer circumference consisted of trophectodermal cells and then, undifferentiated cells migrated into the inside through growth.

### Example 5

In order to study the presence or absence of intestinal cells and cells derived from 3 germ layers in tissues in Example 1, tissues having a sac-shaped structure obtained by culturing cells on a cell culture substrate having annular cell-adhesive parts having an inside diameter of 280 μm or an inside diameter of 380 μm and a width of 60 μm, collecting tissues spontaneously detached therefrom in 3 to 4 weeks after the start of culture, and suspension-culturing the tissues up to 6 weeks after the start of culture in another dish were evaluated by immunostaining.

The tissues were fixed overnight using iPGell (GenoStaff Co., Ltd.) and a 4% paraformaldehyde solution (Wako Pure Chemical Industries, Ltd.) according to the protocol attached to the product. The fixed tissues were embedded in paraffin, and then, tissue sections having a thickness of 4 to 6 μm were produced. Antibody staining was performed by the method described in Uchida et al., JCI Insight, Vol. 2, e86492, 2017. The antibody staining method is as described below.

The tissue sections were incubated overnight at 4° C. with a rabbit IgG-labeled anti-CDX2 antibody (Abcam plc; dilution ratio: 1/1000), a mouse IgG-labeled anti-villin antibody (Santa Cruz Biotechnologies, Inc.; dilution ratio: 1/200), a mouse IgG-labeled anti-smooth muscle actin antibody (Sigma-Aldrich Co. LLC; dilution ratio: 1/500), or a rabbit IgG-labeled anti-PGP9.5 antibody (DAKO; dilution ratio: 1/200) for primary antibody staining. The tissue sections thus stained with the primary antibody were washed with PBS three times for 5 minutes and then incubated at room temperature for 1 hour with an Alexa 488-labeled anti-rabbit IgG antibody or an Alexa 546-labeled anti-mouse IgG antibody (both from Molecular Probes; dilution ratio: 1/1000) diluted with PBS, for secondary antibody staining. The tissue sections thus stained with the secondary antibody were washed with PBS three times for 5 minutes. Thereafter, the nuclei of the cells were stained (DAPI; Sigma-Aldrich Co. LLC; dilution ratio: 1/1000) and mounted.

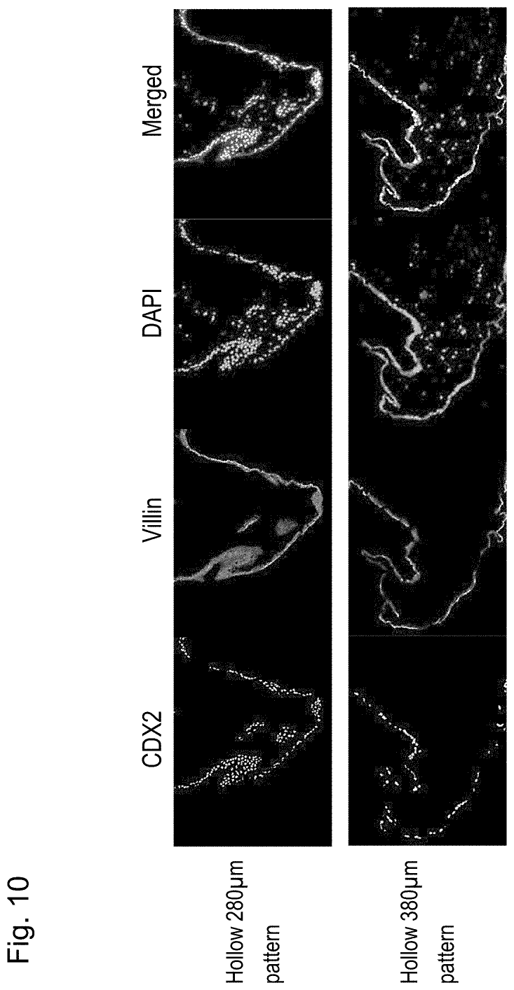

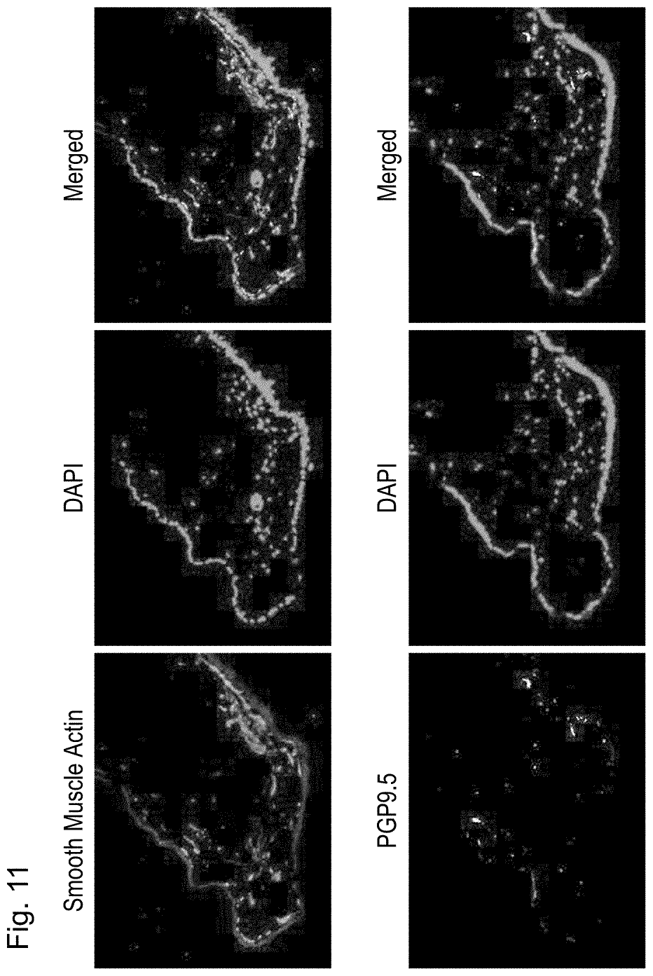

FIG. 10 shows results of staining, with an anti-CDX2 antibody, an anti-villin antibody and DAPI, of tissues formed by culture on the cell culture substrate having annular cell-adhesive parts having an inside diameter of 280 μm or 380 μm and a width of 60 μm in Example 1. The results shown in FIG. 10 show that the tissues formed in Example 1 included intestinal epithelial tissues having CDX2-positive cell nuclei and villin-positive epithelium and having villi. FIG. 11 shows results of staining, with a smooth muscle actin antibody, an anti-PGP9.5 antibody and DAPI, of tissues formed by culture on the cell culture substrate having annular cell-adhesive parts having an inside diameter of 380 μm and a width of 60 μm in Example 1. The results shown in FIG. 11 show that the tissues formed in Example 1 had endoderm-derived intestinal epithelial tissues as well as mesoderm-derived smooth muscle actin-positive muscle tissues or ectoderm-derived PGP9.5-positive nerve fiber-like tissues. The results shown in FIG. 10 and FIG. 11 show that the tissues formed in Example 1 included tissues derived from 3 germ layers.

### Example 6

In order to study an appropriate size of an annular cell-adhesive part and the width of the adhesive part, the following analysis was conducted.

Each cell culture substrate having an annular cell-adhesive part having a different inside diameter and width was produced in the same manner as in Example 1. Edom iPS cells were cultured in the same manner as in Example 1. Tissues having a sac-shaped structure were classified by visual evaluation according to the state of formation into 3 grades: ++(tissues having a sac-shaped structure were efficiently obtained), +(although differentiation into tissues having a sac-shaped structure occurred, many tissues were peeled off, or the generation of the tissues was as slow as that in Comparative Example 1), and—(no tissue was obtained because cells were peeled off or failed to cover by cell growth in the process of culture).

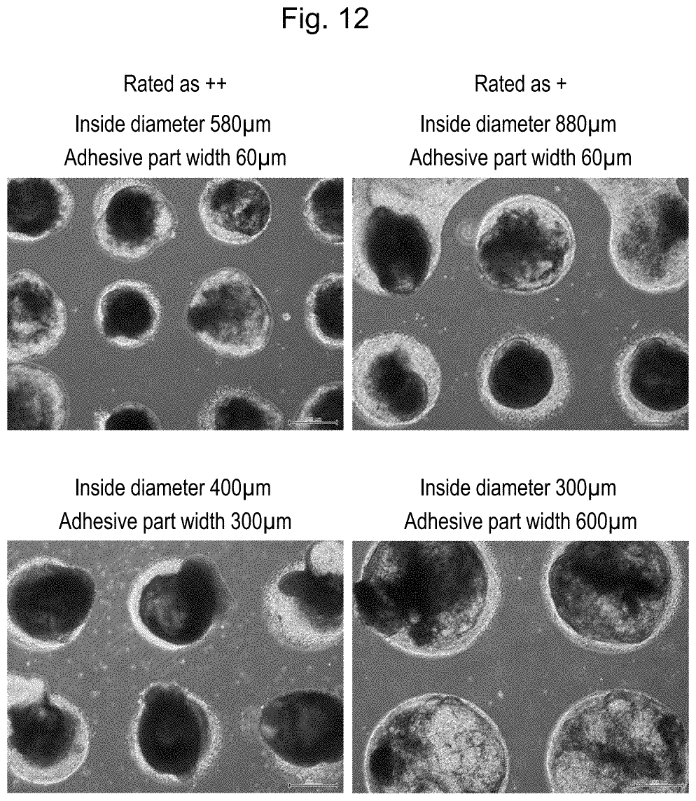

Observation images of typical examples are shown in FIG. 12. FIG. 12 is observation images on day 18 of culture on a cell culture substrate having an annular cell-adhesive part of each size. When rated “++” (left columns), tissues having a sac-shaped structure were able to be detached and collected within 3 weeks.

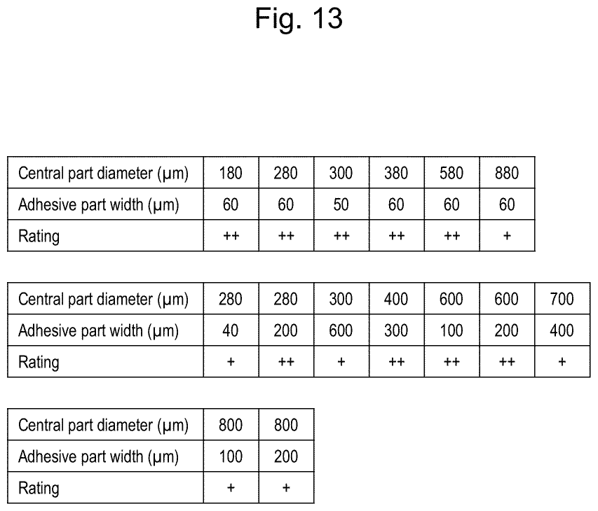

The results are shown in FIG. 13. FIG. 13 also includes the contents of the analysis results in Example 1. These results show that the inside diameter of the annular cell-adhesive part is preferably 180 to 880 μm and more preferably 180 to 600 μm, and show that the width of the annular cell-adhesive part is preferably in a range of 30 to 400 μm, more preferably in a range of 40 to 400 μm, and particularly preferably 60 to 300 μm.

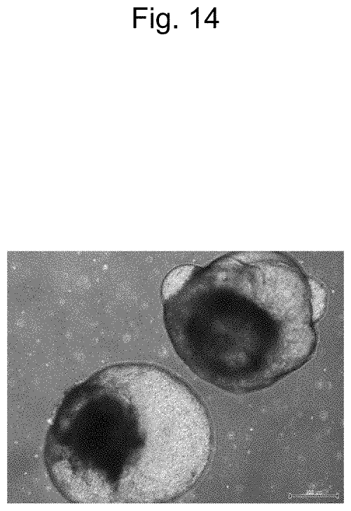

FIG. 14 shows an observation image of a tissue having a sac-shaped structure obtained by culture on a cell culture substrate having an annular cell-adhesive part having an inside diameter of 580 μm and a width of 60 μm.

### Example 7

The following study was conducted on the shape of a cell-adhesive part.

The studied shapes of cell-adhesive parts in cell culture substrates of Example were as shown in FIG. 15A, FIG. 15B, FIG. 15C, and FIG. 15D.

(15A) A square cell-adhesive part having an inside dimension of 280 μm to 300 μm in side and a width of 50 μm to 60 μm
(15B) An annular cell-adhesive part having an inside diameter of 280 μm and a width of 60 μm and lacking ⅛ in the circumferential direction
(15C) A rectangular cell-adhesive part having an inside dimension of 600 μm in long side and 300 μm in short side and a width of 50 μm
(15D) A square cell-adhesive part having an inside dimension of 600 μm in side and a width of 50 μm

Methods for producing these cell culture substrates and a cell culture method are as described in Example 1.

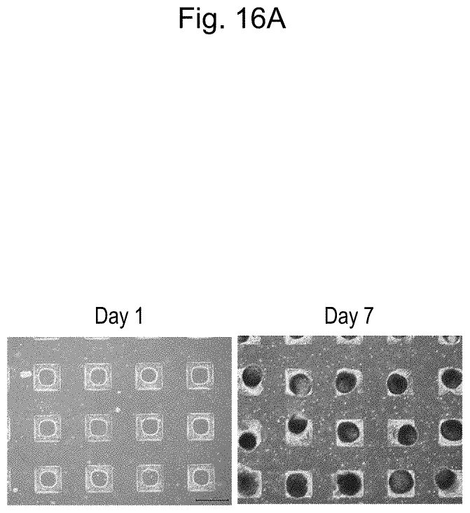

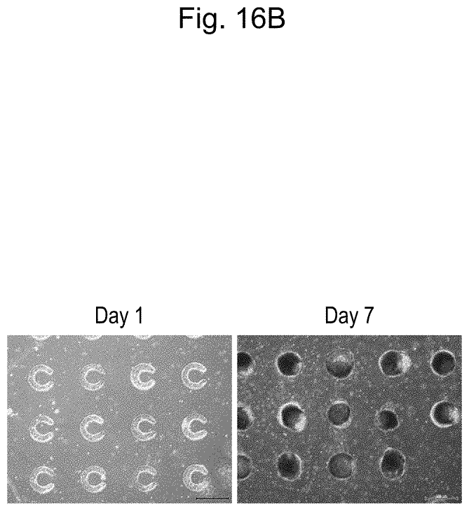

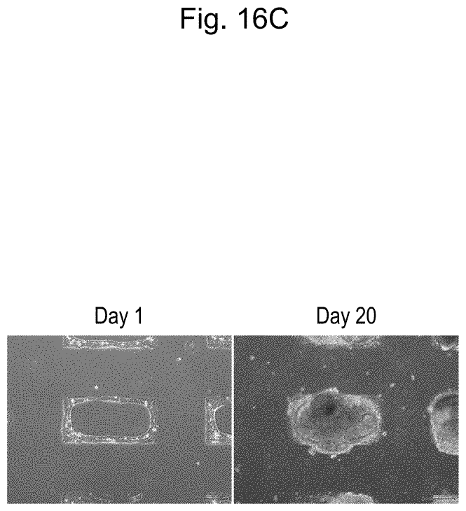

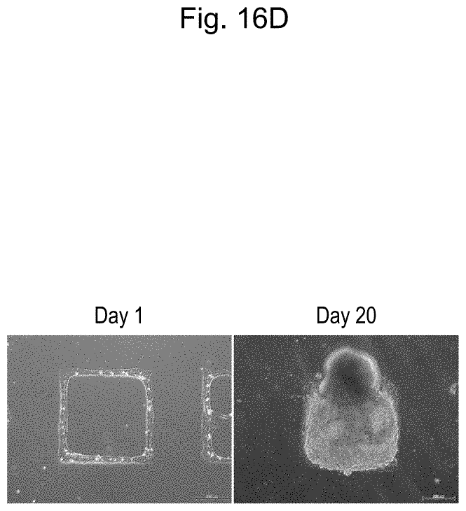

FIGS. 16A, 16B, 16C, and 16D show observation images of cultures obtained by culturing Edom iPS cells using cell culture substrates having cell-adhesive parts in the shapes shown in FIGS. 15A, 15B, 15C, and 15D, respectively. In the case of using the cell culture substrate having any of the shapes of the cell-adhesive parts, tissues having a sac-shaped structure were obtained in a short period, as in the other Examples. Even in the annular cell-adhesive part lacking ⅛ in the circumferential direction shown in FIG. 15B, tissues having a sac-shaped structure were obtained, as in the circumferentially completely annular cell-adhesive part, because the missing part was covered with cells that grew.

From these results, it is evident that the shape of the cell-adhesive part is not particularly limited and is not necessarily required to be round. Furthermore, a closed system can be created by culture, and the initial pattern is not necessarily required to be a closed pattern. Moreover, even a cell-adhesive part having a rectangular shape surrounding a non-cell-adhesive part also yielded a gut structure, suggesting that the non-cell-adhesive part surrounded by the cell-adhesive part does not have to have equal vertical and horizontal lengths and the non-cell-adhesive part surrounded by the cell-adhesive part may be oval or semicircular in shape.

### COMPARATIVE EXAMPLES

The following study was conducted on the shape of a cell-adhesive part in a cell culture substrate.

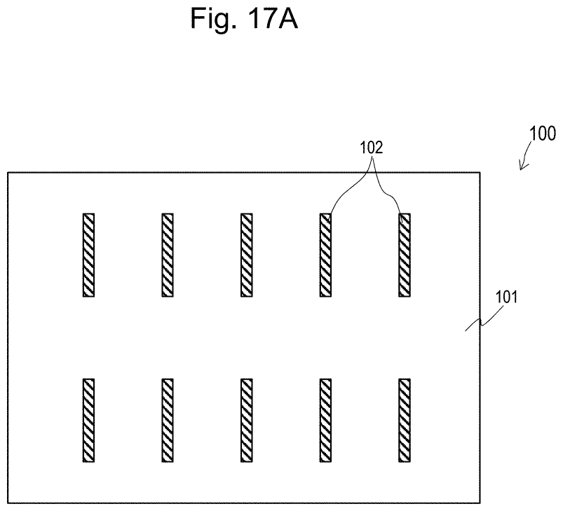

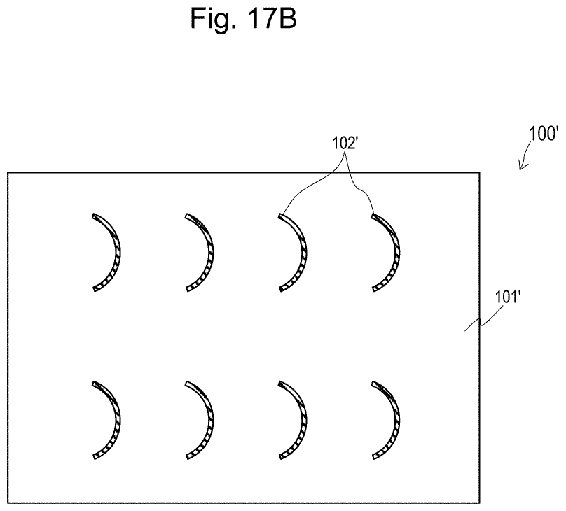

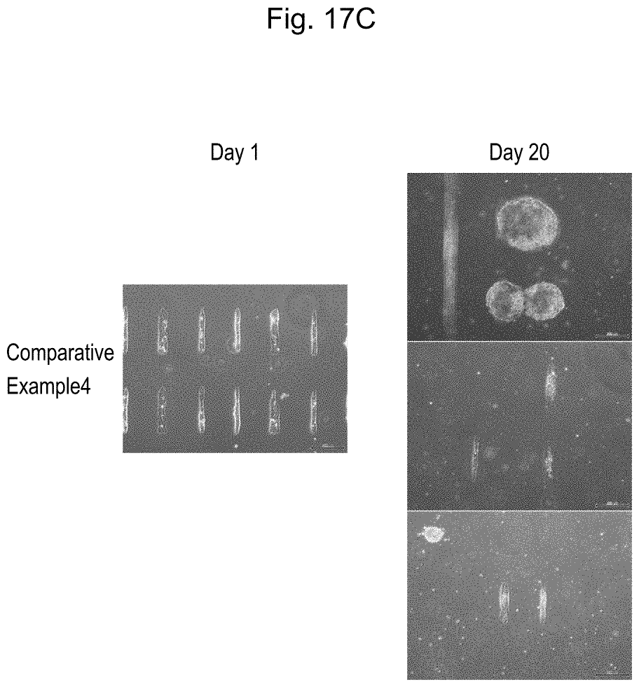

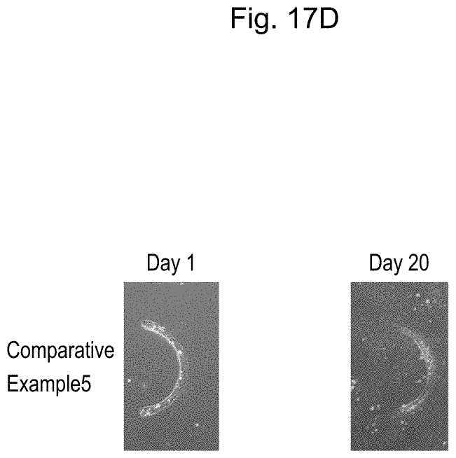

As a cell culture substrate of Comparative Example 4, cell culture substrate 100, shown in FIG. 17A, having a surface having non-cell-adhesive region 101, and a plurality of linear cell-adhesive regions 102 of 30 to 50 μm in width arranged in parallel at intervals of 200 μm in the non-cell-adhesive region 101 was used.

As a cell culture substrate of Comparative Example 5, cell culture substrate 100′, shown in FIG. 17B, having a surface having non-cell-adhesive region 101′, and a plurality of arc-like cell-adhesive regions 102′ of 50 μm in width lacking over a half in the circumferential direction of a ring having an inside diameter of 600 μm arranged in the non-cell-adhesive region 101′ was used.

Methods for producing the cell culture substrate 100 of Comparative Example 4 and the cell culture substrate 100′ of Comparative Example 5 are as described in Example 1.

Cell culture was performed under the same conditions as in Example 1 on the cell culture substrate 100 of Comparative Example 4 and the cell culture substrate 100′ of Comparative Example 5.

FIG. 17C shows photographs on days 1 and 20 of cell culture on cell culture substrate 100 in Comparative Example 4. As in the upper photograph of day 20 in FIG. 17C, there was a case in which tissues formed by adhering to a pair of adjacent cell-adhesive regions 102 were fused to form one sac-shaped cell construct, or in which a tissue formed by adhering to one cell-adhesive region 102 formed one sac-shaped cell construct in itself. However, in many cases, as in the middle or lower photograph of day 20 in FIG. 17C, no sac-shaped cell construct was formed on the cell culture substrate 100 of Comparative Example 4.

FIG. 17D shows photographs on days 1 and 20 of cell culture on cell culture substrate 100′ in Comparative Example 5. As in the photograph of day 20 in FIG. 17D, no sac-shaped cell construct was formed on the cell culture substrate 100′ of Comparative Example 5. In the cell culture substrate 100′ of Comparative Example 5, tissues that attached and grew on the arc-like cell-adhesive regions 102′ failed to form a closed cell construct. By contrast, on cell culture substrate 1 having cell-adhesive parts 22 intermittently surrounding the non-cell-adhesive parts (central parts) 21 shown in FIG. 15B, tissues that attached and grew on the cell-adhesive parts 22 were able to form closed cell constructs so as to span the missing portions in the cell-adhesive parts 22. Thus, it can be concluded that a cell culture substrate having a structure where a cell-adhesive part is arranged so as to surround a central part serving as a non-cell-adhesive part and a cell accumulation of interest can be formed around the central part can attain the object to obtain a sac structure.

### Example 8

In Example 1, the non-cell-adhesive part was formed by coating with polyethylene glycol. In this Example, whether other compounds used instead of polyethylene glycol could produce similar effects was studied. Accordingly, a cell culture substrate having a plurality of annular cell-adhesive parts having an inside diameter of 280 μm or 380 μm and a width of 60 μm was produced by the following method.

Glass (170 μm thick) was cut into 125 mm square as a substrate, immersed in an alkali washing solution PARKEM (Parker Corp., PK-LCG23) for 48 hours or longer for preliminary washing, and rinsed with pure water. Thereafter, the substrate was irradiated with vacuum ultraviolet ray (172 nm) for 6 minutes in a nitrogen atmosphere. Next, as a process of forming an annular pattern, a photosensitive dry film resist (Nichigo-Morton Co., Ltd., NIT915) was laminated onto the washed glass substrate on a hot plate of 100° C., and heated and kept for 5 minutes. Thereafter, the substrate was irradiated with 200 mJ of UV light (broad band) via a photomask having openings of the same size as that of annular patterns of the size described above. Resist patterns were formed by treatment with an aqueous sodium carbonate solution for 2 minutes, followed by baking at 100° C. for 5 minutes and subsequent step baking at 180° C. for 5 minutes. The substrate thus treated was cut into 15 mm×25 mm square. Aside from this, 0.5 wt % Lipidure® (NOF Corp.) was dissolved in 99.5% ethanol to prepare a solution, which was then used in an amount on the order of 200 μl to coat the substrate cut out by cast coating. The substrate was naturally dried for 1 day, then immersed in AZ Remover 100 (Tokyo Ohka Kogyo Co., Ltd.) under ultrasonic application for 5 minutes for resist removal, and then rinsed. Finally, EOG sterilization treatment was performed for 22 hours. Thus, a substrate having a plurality of annular cell-adhesive parts having an inside diameter of 280 μm or 380 μm and a width of 60 μm where the surface of the glass substrate was exposed, and non-cell-adhesive parts where the surface (inside and outside the annular cell-adhesive part) of the glass substrate was coated with Lipidure®, was obtained. The cell culture substrate in this Example has a cross-sectional structure as shown in FIG. 1(B), as in Example 1.

The substrate was cut into a size of 15 mm×25 mm and studied by seeding iPS cells, as in Example 1.

The film thicknesses of the Lipidure® coatings constituting the non-cell-adhesive parts on the substrate were measured in a profilometer and were consequently 288 nm on average.

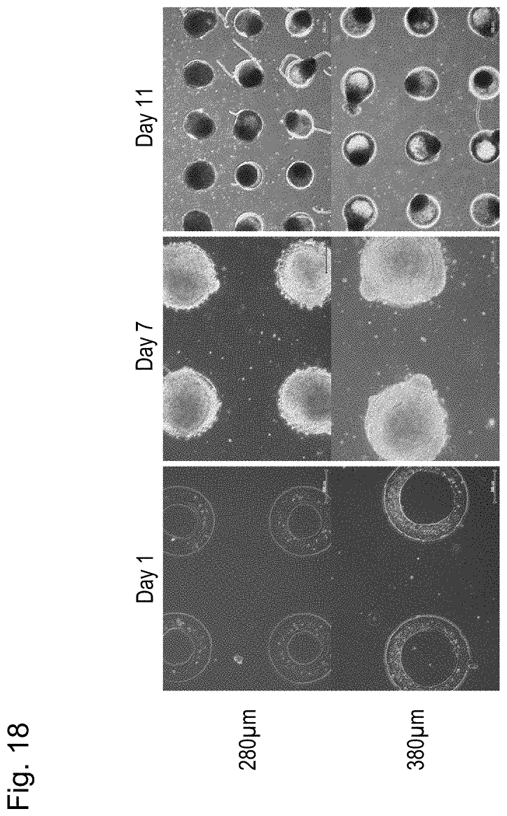

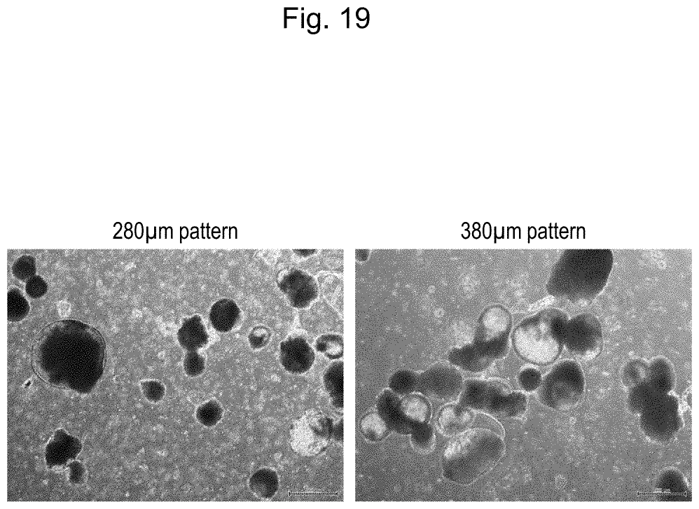

FIG. 18 shows observation images on culture days 1, 7 and 11 of a culture on each substrate. The magnification factors of the observation images on culture days 1 and 7 are higher than that of the observation image on culture day 11. FIG. 19 shows tissues having a sac-shaped structure obtained by culture for 3 weeks.

In Example 1 or other examples, the non-cell-adhesive parts were prepared by coating a substrate surface with polyethylene glycol, a compound suppressing cell adhesion. The results of this experiment show that in the case of using a substance other than polyethylene glycol as a compound suppressing cell adhesion, a non-cell-adhesive part can also be formed, as in polyethylene glycol.

### Example 9

Cell culture substrates having a plurality of annular cell-adhesive parts having a total of 4 sizes (inside diameter: 600 μm or 800 μM, width: 100 μm or 200 μm) used in Example 6 were produced and prepared in the same manner as in Example 1. Each of these four cell culture substrates of Example was cut into 5 cm square, and this square piece was disposed in a round dish for cell culture of 10 cm in diameter.

In Comparative Example, a cell culture substrate having a plurality of round cell-adhesive parts of 1500 μm in diameter of Comparative Example 1 was prepared and also cut into 5 cm square, and this square piece was disposed in a round dish for cell culture of 10 cm in diameter.

As cells to be cultured, iPS cells (KAC Co., Ltd.) established from human T cells, and iPS cells (Nippon Genetics Co., Ltd.) established from human fibroblasts were used.

The cells described above were seeded at a density of 1×10⁷ cells to the dish for cell culture in which each of the cell culture substrates was disposed, and cultured under the conditions described in Example 1.

The tissue collection rate of a sac-shaped structure on culture day 60 was 0.1% for culture on the cell culture substrate of Comparative Example, and by contrast, was 1.0% or more for culture on the four cell culture substrates of Example, irrespective of whether either of the above-described cells was cultured. Improvement in yield was confirmed in the latter.

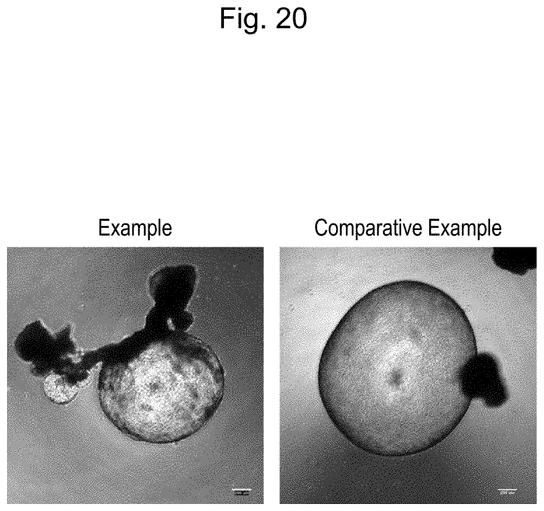

The photograph of “Example” in FIG. 20 is a typical photograph of a sac-shaped cell construct formed by culturing iPS cells (Nippon Genetics Co., Ltd.) established from human fibroblasts on a cell culture substrate having a plurality of annular cell-adhesive parts having a size of 600 μm in inside diameter and 100 μm in width. The photograph of “Comparative Example” in FIG. 20 is a typical photograph of a sac-shaped cell construct formed by culturing the cells on a cell culture substrate of Comparative Example 1 having a plurality of round cell-adhesive parts of 1500 μm in diameter. Each photograph shown in FIG. 20 was taken under a confocal microscope (Leica Camera AG).

From these results, in the case of culturing different cell types, a sac-shaped cell construct was obtained at a high tissue collection rate by culture on a cell culture substrate having an annular cell-adhesive part according to an embodiment of the present disclosure as compared with culture on an existing cell culture substrate having a round cell-adhesive part. In this Example compared with Example 1 and other examples, the tissue collection rate tended to be low. However, since the tissue collection rate was also low in Comparative Example, the low tissue collection rate in this Example was presumably attributed to a feature of differentiation induction of cells.

### Example 10

Experimental results of culturing cancer cells on cell culture substrates having different shapes of a cell-adhesive part to induce sac-shaped cell constructs (cyst tissues) will be given below.

As cell culture substrates, cell culture substrates having the following shapes of cell-adhesive parts were used.

(Shape 1) An annular cell-adhesive part having an inside diameter of 280 μm and a width of 60 μm (see FIG. 1)
(Shape 2) A semicircular arc-like cell-adhesive part having an inside diameter of 280 μm and a width of 60 μm and lacking ½ in the circumferential direction of a ring (see FIG. 22)
(Shape 3) A square cell-adhesive part having an inside dimension of 280 μm in side and a width of 60 μm (see FIG. 15A)
(Shape 4) A C-shaped cell-adhesive part having an inside diameter of 280 μm and a width of 60 μm and lacking ⅛ in the circumferential direction of a ring (see FIG. 15B)

Methods for producing these cell culture substrates are as described in Example 1.

As cancer cells, large intestinal epithelial cancer-derived Caco-2 cells were used. Cell culture was performed by the following procedures.

Caco-2 cells were allowed to grow in a cell culture dish of 10 cm in diameter (Corning Inc.) using DMEM medium (Sigma-Aldrich Co. LLC) containing 10% fetal bovine serum (FBS) and 1% Glutamax (Life Technologies Corp.). After reaching about 80% confluency, the cells were detached by treatment with 0.25% trypsin-EDTA solution (FUJIFILM Wako Pure Chemical Corp.) for 2 minutes. Then, the detached cells were seeded at the same cell density as in Example 9 to a round cell culture dish of 10 cm in diameter in which each square cell culture substrate of 5 cm square having a plurality of cell-adhesive parts (having any of the shapes 1 to 4) on a glass substrate was disposed on the inside bottom surface. The cells were cultured. The medium used was the same as the medium used in the growth of the Caco-2 cells. Precoating for cell adhesion was not carried out for the cell culture substrate. The whole amount of the medium was replaced once every 2 to 3 days. The cells were maintained for 18 days, and the presence or absence of formation of a sac-shaped cell construct was examined.

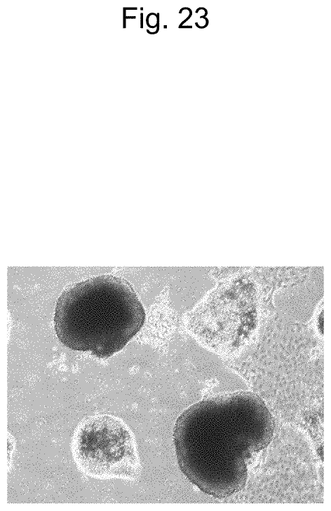

FIG. 23 shows an observation image of a culture on culture day 18 obtained by culturing Caco-2 cells on a cell culture substrate having a cell-adhesive part having the shape 1 described above.

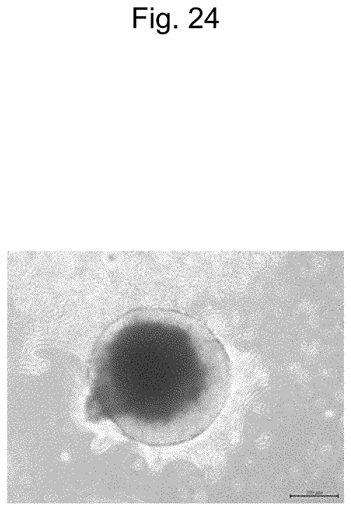

FIG. 24 shows an observation image of a culture on culture day 18 obtained by culturing Caco-2 cells on a cell culture substrate having a cell-adhesive part having the shape 2 described above.

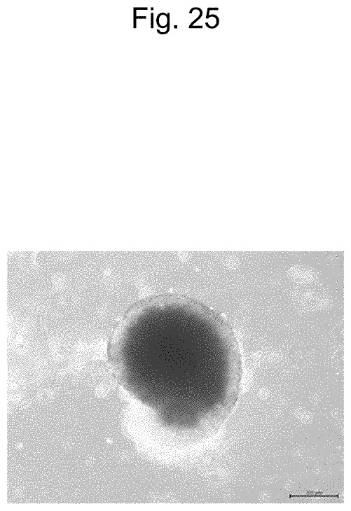

FIG. 25 shows an observation image of a culture on culture day 18 obtained by culturing Caco-2 cells on a cell culture substrate having a cell-adhesive part having the shape 3 described above.

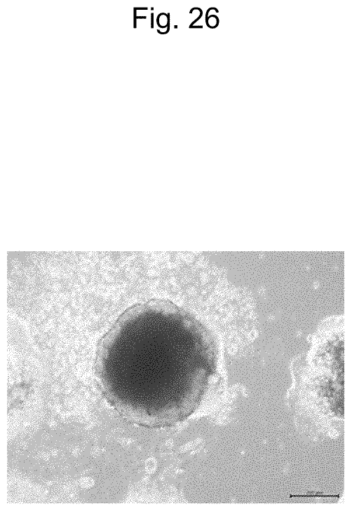

FIG. 26 shows an observation image of a culture on culture day 18 obtained by culturing Caco-2 cells on a cell culture substrate having a cell-adhesive part having the shape 4 described above.

In the case of using the cell culture substrate having any shape of the cell-adhesive part, a sac-shaped cell construct was able to be induced from Caco-2 cells.

Although not shown, no sac-shaped cell construct was obtained by culturing large intestinal epithelial cancer-derived Caco-2 cells under the same conditions as above except that a cell culture substrate having a plurality of round cell-adhesive parts of 1500 μm in diameter described in Comparative Example 1 was used.

All publications, patents and patent applications cited herein are incorporated herein by reference in their entirety.

1. A cell culture substrate having a surface comprising a cell culture part, wherein
the cell culture part comprises a non-cell-adhesive part, and a cell-adhesive part extending continuously or intermittently along the periphery of the non-cell-adhesive part and surrounding the non-cell-adhesive part.

2. The cell culture substrate according to claim 1, wherein
the cell culture substrate comprises a support substrate having a surface comprising
a first non-cell-adhesive part, and
the one or more cell culture parts arranged in the first non-cell-adhesive part, wherein
each of the one or more cell culture parts comprises a central part serving as a second non-cell-adhesive part, and the cell-adhesive part extending continuously or intermittently along the periphery of the central part and surrounding the central part, wherein a cell-adhesive part is not present in the second non-cell-adhesive part.

3. The cell culture substrate according to claim 1, wherein a distance between two points of intersection of a straight line with an inner circumference of the cell-adhesive part is larger than 80 μm and 880 μm or smaller, the straight line passing through a midpoint between two points on the inner circumference of the cell-adhesive part opposed to and most distant from each other across the non-cell-adhesive part.

4. The cell culture substrate according to claim 1, wherein a width of the cell-adhesive part along a straight line is larger than 30 μm and 400 μm or smaller, the straight line passing through a midpoint between two points on an inner circumference of the cell-adhesive part opposed to and most distant from each other across the non-cell-adhesive part.

5. The cell culture substrate according to claim 1, wherein the non-cell-adhesive part is a surface coated with a layer comprising a hydrophilic polymer.

6. The cell culture substrate according to claim 5, wherein the hydrophilic polymer is one or more hydrophilic polymers selected from polyalkylene glycol and a zwitterionic polymer having a phospholipid polar group.

7. The cell culture substrate according to claim 6, wherein the polyalkylene glycol is polyethylene glycol.

8. The cell culture substrate according to claim 1, wherein the cell culture substrate comprises a glass substrate as a support substrate.

9. A kit for cell culture comprising a cell culture substrate according to claim 1.

10. The kit according to claim 9, further comprising one or more members selected from a medium and a precoating treatment agent.

11. A production method comprising:
seeding cells onto the cell culture substrate according to claim 1, and
culturing the cells seeded to induce a sac-shaped cell construct.

12. The method according to claim 11, wherein the cells are stem cells or cancer cells.

13. A sac-shaped cell construct, produced by the method according to claim 11.
